(19) United States
(12) Patent Application Publication
(10) Pub. No.: US 2003/0144236 A1
(43) Pub. Date: Jul. 31, 2003

(54) Novel specific inhibitor of the cyclin kinase inhibitor p21 (wafl/cip1)

(76) Inventor: Robert H Weiss, Vacaville, CA (US)

Correspondence Address: MANDEL & ADRIANO, 55 SOUTH LAKE AVENUE, SUITE 710, PASADENA, CA, 91101, US

(21) Appl. No.: 10/240,140

(22) PCT Filed: Mar. 29, 2001
(86) PCT No.: PCT/US01/10443

(51) Int. Cl. A61K 48/00; A61K 39/395

(52) U.S. Cl. 514/44; 424/146.1

(57) ABSTRACT

Methods and compositions for regulating abnormal cell growth and proliferation mediated by p21^{Waf1/Cip1} using inhibitors of p21^{Waf1/CiP1}.

This application is based on and claims the priority of U.S. Serial No. 60/193,155, filed Mar. 29, 2000, the contents of which are hereby incorporated by reference in their entirety.

Throughout this application various publications are referenced. The disclosures of these publications, in their entireties, are hereby incorporated by reference into this application, in order to more fully describe the state of the art to which this invention pertains.

### FIELD OF INVENTION

The present invention relates to methods and compositions for regulating cell growth and proliferation, mediated by cyclin-dependent kinases, by inhibiting p21^{Waf1/Cip1}, and more particularly to the prevention and treatment of diseases associated with abnormal proliferation of cells, using p21^{Waf1/Cip1} inhibitory agents.

### BACKGROUND OF THE INVENTION

The mechanism by which cells “decide” whether to grow or not to grow is of paramount importance in a surprising variety of diseases. Unregulated or abnormal cell growth in vascular smooth muscle (VSM) and phenotypically-related glomerular mesangial cells (Dubey R K, et al. Curr Opin Nephrol Hypertens 6:88-105, 1997), is the underlying pathogenic mechanism in such diverse diseases as hemodialysis graft stenosis, angioplasty restenosis (Ross, 1993 Nature, 362, p. 801-809), and atherosclerosis, as well as in mesangial proliferative kidney disease (Megyesi et al., 1999 Proc. Natl. Acad. Sci. U.S.A., 96, p. 10830-10835). It has been suggested that pharmacological methods to increase p21^{Waf1/Cip1} may be useful in preventing the VSM cell proliferation seen after coronary angioplasty (Kusama et al., 1999 Atherosclerosis, 143, p. 307-313; Takahashi et al., 1999 Circ. Res., 84, p. 543-550; and Yang et al., 1996: Semin. Interv. Cardiol., 1, p. 181-184). Thus, research directed at understanding the mechanisms by which these processes occur in VSM cells is critical to the development of specific therapies for these diseases.

Most likely due to the fact that protection from these often deadly diseases has provided significant survival advantage over evolutionary time, organisms have evolved complex and often redundant systems for keeping these essential, but potentially lethal, cellular processes, such as unregulated cell growth, in check. Consequently, there exist cell cycle regulators at multiple levels of the cell growth hierarchy: from the growth factor receptor regulatory proteins (such as receptor phosphorylation events and various G-proteins), through the cytoplasmic signal protein interactions (such as the mitogen-activated protein serine/threonine kinase (MAPK), stress-activated protein kinase (SAPK), and Janus family of protein kinase-signal transducers and activators of transcription (JAK-STAT) systems [Frye, R. A. in Oncogenes and Tumor Suppressor Genes in Human Malignancies, Benz C C and Liu, E T, eds. 63:281-299, Kluwer Academic Publishers, Boston], to the nuclear transcriptional control machinery (such as the cyclins, cyclin kinases, and cyclin kinase inhibitors) (Lavoie J N, et al. Prog Cell Cycle Res 2:49-58, 1996).

Mechanisms and regulation of the cyclin kinase system in VSM cells have been studied. These molecules (such as the cyclins, cyclin kinases, and cyclin kinase inhibitors), which regulate cell growth, are very distal along the growth factor signaling pathways, and may, therefore, be among the ultimate arbiters of the decision a cell must make whether it will proceed through the cell cycle and lead to the production of cellular progeny or die. Since many growth-controlling signaling pathways converge on the cyclin system, elucidating the mechanism of regulation of these molecules will likely lead to the development of pharmaceuticals which target these molecules and, consequently, are useful for the treatment of vascular and renal diseases as well as cancer.

Cell cycle progression is finely regulated by the interplay between the cyclin-dependent kinases (cdks) and the cdk inhibitors (CKIs). Cyclin is a protein involved in the cell cycle that accumulates during interphase and is destroyed during mitosis. Cdks are a well-conserved family of serine/threonine protein kinases, found in yeast and in at least eight different animal cells, which function in mitogenic signaling through their activation by the cyclins. This in turn leads to a cascade of events whereby the mitogen-stimulated cyclin D-dependent kinases phosphorylate retinoblastoma protein (Rb), causing release of inhibition of the transcription factor family known as E2F, and allowing S-phase specific gene transcription and subsequent progression through the G1/S transition (Sherr and Roberts, 1999, Genes and Dev., 13, p. 1501-1512).

The Cip/Kip family of CKIs (p21^{Waf1/Cip1}, p27^{Kip1}, and p57^{Kip2}) regulate the activity of the cyclin/cdk complex and have been shown to negatively regulate the process of cyclin-mediated cell cycle progression through inhibition of the cdks (p21^{Waf1/Cip1} (Gu et al. 1993, Nature, 366, 707-710; Harper et al., 1993, Cell, 75, 387-400; El-Deiry et al. 1993, Cell, 75, 817-825; Xiong et al. 1993, Nature, 366, 701-704; Dulic et al. 1994, Cell, 76, 1013-1023; Noda et al. 1994, Exp. Cell Res., 211, 90-98), p27^{Kip1} (Polyak et al. 1994, Genes & Dev. 8, 9-22; Polyak et al. 1994, Cell, 78, 59-66; and Toyoshima and Hunter, 1994, Cell, 78, 67-74), and p57^{Kip2} (Lee et al. 1995, Genes & Dev. 9, 639-649; and Matsuoka et al. 1995, Genes & Dev. 9, 650-662)).

The protein p21^{Waf1/Cip1} was first described in 1992 (Xiong et al., 1992 Cell, 71, p. 505-514). The sequences of the human, rat and mouse p21^{Waf1/Cip1} genes are known (GenBank entries CAB06656, I84725 and I49023, respectively), and polyclonal and monoclonal antibodies, to human and rodent species, are commercially available. The human protein has been expressed in E. coli by commercial sources (Santa Cruz Biotechnology, Santa Cruz, Calif.).

The net result of induction or overexpression of the CKIs (particularly those in the Cip/Kip family) generally is cell cycle inhibition and growth suppression in VSM and other cell types (Chang et al., 1995 J. Clin. Invest., 96, p. 2260-2268; Ishida et al., 1997 J.Biol.Chem., 272, p. 10050-10057; Matsushita et al., 1998 Hypertension, 31, p. 493-498; Sewing et al., 1997 Mol. Cell Biol., 17, p. 5588-5597; and Weiss et al., 1999 J. Am. Soc. Nephrol., 9, p. 1880-1890). Consistent with this, the CKIs are down-regulated in response to a variety of mitogens, and overexpression of these molecules leads to growth arrest (Kato et al., 1994 Cell, 79, p. 487-496; Nourse et al., 1994 Nature, 372, p. 570-573; Pagano et al., 1995 Science, 269, p. 682-685; and Resnitzky et al., 1995 Mol. Cell Biol., 15, p. 4347-4352).

While much of the early work on the CKIs has focused on their role as growth inhibitors, it had been somewhat puzzling that expression of these molecules increases early after mitogen stimulation (Depoortere et al., 1996 J. Cell Sci., 109 (Pt 7), p. 1759-1764; and Michieli et al., 1994 Cancer Res., 54, p. 3391-3395). CKIs have also been implicated in positive effects on cyclin/cdk activitation (Cheng et al., 1998 Proc. Natl. Acad. Sci.U.S.A., 95, p. 1091-1096; LaBaer et al., 1997 Genes Dev., 11, p. 847-862; and Zhang et al., 1994 Genes Dev., 8, p. 1750-1758). This led to more recent data showing the ability of some CKIs to take part in formation of the cyclin/cdk complexes, and thus to serve as “assembly factors” important for promoting cyclin/cdk association (Hiyama et al., 1998 Oncogene, 16, p. 1513-1523; and LaBaer et al., 1997 Genes Dev., 11, p. 847-862). Various CKIs have also been reported to act as “assembly factors” in other cells, both in vivo (Cheng et al., 1999 EMBO J., 18, p. 1571-1583) and in vitro (LaBaer et al., 1997: Genes Dev., 11, p. 847-862). In support of this role for the CKIs, others have shown that assembly of cyclin D1/D2-cdk4 complexes was impaired in fibroblasts from mice lacking the p21^{Waf1/Cip1} and/or p27^{Kip1} genes (Cheng et al., 1999 EMBO J., 18, p. 1571-1583), and that both p21^{Waf1/Cip1} and p27^{Kip1} actively promoted interaction between the cyclin Ds and their counterpart cdks by stabilizing this complex (LaBaer et al., 1997 Genes Dev., 11, p. 847-862). However, primary fibroblasts from p21- and p27-null mice did not show overtly abnormal cell cycles, despite the finding by those investigators that overall cyclin D-dependent kinase activity was reduced below the assay limit of detectability. Previous studies have not shown inhibition of growth with interference of cyclin/cdk association. The cyclin D1/cdk 4 interaction occurs early after growth factor stimulation (reviewed in Arellano and Moreno, 1997 Int. J. Biochem. Cell Biol., 29, p. 559-573) and this interaction is facilitated by p21^{Waf1/Cip1} and p27^{Kip1} in vivo (LaBaer et al., 1997: Genes Dev., 11, p. 847-862).

Thus, CKIs exhibit both positive and negative effects on growth and apoptosis in a variety of cell types, including VSM cells. A further example of this is the mechanism of action of the HMG CoA reductase inhibitors, where accelerated graft atherosclerosis in heart, and probably renal, transplant patients is attenuated by the statins (Katznelson S, et al. Transplantation 61:1469-1474, 1996; and Southworth M R, Mauro V F. Ann Pharmacol 31:489-491, 1997). Many of the statins have been shown to attenuate smooth muscle growth and promote apoptosis in association with an increase in the cyclin kinase inhibitors p21 and p27 (Baetta R, et al. Pharmacol Res 36:115-121, 1997; Terada Y, et al. J. Am. Soc Nephrol 9:2235-2243, 1999; Weiss R H, et al. J Am Soc Nephrol 9:1880-1890, 1999; and Laufs U. et al. J Biol Chem 274:21926-21931, 1999), although whether this is the mechanism of this effect is unknown. Tumor cells that are p21 (−/−) are also known to be sensitized to apoptosis (Stewart Z A, et al. Cancer Res 59:3831-3837, 1999; Fan S et al. Oncogene 14:2127-2136, 1997). What constitutes the “switch” from positive to negative effects of the cyclin kinase inhibitors on both growth and apoptosis is unclear.

There is a need for improved therapies of diseases, associated with abnormal cell growth and proliferation, that take into account the various pathways that result in stimulation of growth and of cells. For the first time, it is shown in the present invention that p21^{Waf1/Cip1} serves a permissive role in platelet-derived growth factor (PDGF)-mediated VSM cell proliferation, such that its presence is required for the mitogenic effect of PDGF. It is thus possible to devise therapeutic strategies to inhibit cell proliferation, in proliferative diseases, by controlling the expression of CKIs, in particular p21^{Waf1/Cip1}.

### SUMMARY OF THE INVENTION

Accordingly, the present invention provides novel methods and compositions for regulating cell growth and proliferation, and treating diseases associated with abnormal cell growth and proliferation, mediated by cyclin-dependent kinases, by inhibiting p21^{Waf1/Cip1} in cells expressing p21^{Waf1/Cip1}, using p21^{Waf1/Cip1} inhibitory agents. The methods are also for preventing and treating fibrotic diseases associated with abnormal cell growth and proliferation. The methods, further include, inhibiting angiogenesis and tumor growth by inhibiting p21^{Waf1/Cip1} in cells expressing p21^{Waf1/Cip1}, using p21^{Waf1/Cip1} inhibitory agents. The methods include using inhibitory agents such as an antisense oligonucleotide of p21^{Waf1/Cip1} and anti-p21^{Waf1/Cip1} antibodies, to prevent transcription and expression of p21^{Waf1/Cip1}.

The therapeutic methods of the invention can also be used in conjunction with radiation therapy and chemotherapy.

### BRIEF DESCRIPTION OF THE FIGURES

FIGS. 1A-D are bar graphs showing that antisense p21^{Waf1/Cip1} oligodeoxynucleotide transfection inhibits VSM cell DNA synthesis in a dose-dependent manner, as described in Example I, infra. A10 VSM cells were lipofected with (A) no DNA, 200 nM of sense p21^{Waf1/Cip1} or antisense p21^{Waf1/Cip1}; and (B) 200 nM of random sequence control oligodeoxynucleotide or antisense p21^{Waf1/Cip1}; and various concentrations of sense p21^{Waf1/Cip1} or antisense p21^{Waf1/Cip1} in (C) A10 and (D) bovine VSM cells. The experiments shown are representative of two to three separate experiments.

FIG. 2 is a bar graph showing VSM cell proliferation is inhibited by antisense p21^{Waf1/Cip1} oligodeoxynucleotide, as described in Example I, infra. A10 VSM cells were transfected as in FIG. 1C. Cell numbers are expressed as mean±s.e.m. of three wells per data point.

FIGS. 3A and B show photographs of antisense oligodeoxynucleotides which were successfully transfected into VSM cells, as described in Example I, infra. FITC-tagged p21^{Waf1/Cip1} antisense oligodeoxynucleotide was lipofected into A10 VSM cells and the same microscopic field was visualized by (A) visible and (B) fluorescence light at 40×.

FIG. 4 depicts a gel showing PMA induces p21^{Waf1/Cip1} in VSM cells, as described in Example I, infra. Non-transfected A10 VSM cells were stimulated with PMA (100 ng/ml) for the times indicated and Western blotted with p21^{Waf1/Cip1} antibody. The experiment shown is representative of three separate experiments.

FIG. 5 depicts a gel showing PMA stimulated CKI induction is blunted in antisense p21^{Waf1/Cip1} transfected cells, as described in Example I, infra. A10 VSM cells were transfected with antisense p21^{Waf1/Cip1} or sense p21^{Waf1/Cip1}, oligodeoxynucleotide as in FIG. 1. After overnight incubation, the cells were stimulated with PMA for the times indicated, lysed, and the lysates were Western blotted with p21^{Waf1/Cip1} antibody. The experiment shown is representative of three separate experiments.

FIGS. 6A and B are gels showing antisense p21^{Waf1/Cip1} inhibition of p21^{Waf1/Cip1}, as described in Example I, infra. A10 VSM Cells were lipofected with antisense p21^{Waf1/Cip1} or sense p21^{Waf1/Cip1} oligonucleotides, as in FIG. 1. (A) antibody to p21^{Waf1/Cip1}; antibody to p27^{Kip1} (B) α-actin antibody. The experiments shown are each representative of two separate experiments.

FIG. 7 depicts a gel showing antisense p21^{Waf1/Cip1} inhibits cyclin D1/cdk4, but not cyclin E/cdk2, association, as described in Example I, infra. A10 VSM cells were transfected with antisense or sense p21^{Waf1/CiP1} as in FIG. 1. The arrowhead indicates cdk 4 or cdk 2. The band to the right of each blot is the 2 h sense lysate immunoprecipitated and immunoblotted with cdk 2 or cdk 4 as a positive control. The thick band at the top of each blot is the heavy chain of IgG from the immunoprecipitation. The experiments shown are each representative of two separate experiments.

FIG. 8 is a gel depicting PMA stimulated CKI induction is blunted in antisense p21^{Waf1/Cip1} transfected cells, as described in Example II, infra. The experiments shown are representative of two separate experiments.

FIGS. 9A-D are bar graphs showing Antisense p21^{Waf1/Cip1} oligodeoxynucleotide has no significant effect DNA synthesis in PMA-inhibitable A431 cells, as described in Example II, infra. A431 or A10 cells were lipofected with from 0 to 400 nM of sense p21^{Waf1/Cip1} or antisense p21^{Waf1/Cip1}. A431 cells were placed in serum-free medium overnight and then stimulated with (A) PDGF-BB (30 ng/ml), (B) 10% serum-containing medium, or (C) PDGF-BB or PMA (100 ng/ml) for another 8 h before [³H]-thymidine was added for overnight incubation. (D) A10 VSM cells were transfected with sense and antisense p21^{Waf1/Cip1} oligonucleotides as above and treated similarly to (B). DNA synthesis was assessed by [³H]-thymidine incorporation and is expressed as mean±s.e.m. of three wells per data point. The absolute counts differ between experiments due to different confluency of the cells. The experiments shown are representative of two separate experiments.

FIG. 10 is a Western blot showing levels of p53 protein were not altered in A431 cells as compared to A10 VSM cells, as described in Example II, infra. The experiment shown is representative of two separate experiments.

FIG. 11 is a Western blot showing Serum-induced hyperphosphorylation of Rb was not altered in A431 cells, as described in Example II, infra. The experiment shown is representative of two separate experiments.

FIG. 12 is a gel showing Antisense p21^{Waf1/Cip1} altered cyclin D1/cdk4 association in A431 cells, as described in Example II, infra. The arrowhead indicates cdk4 (top blot) or cdk2 (bottom blot). The band to the right of each blot is the 2 h sense lysate immunoprecipitated and immunoblotted with cdk2 or cdk4 as a positive control. The thick band at the top of each blot is the heavy chain of IgG from the immunoprecipitation. The experiments shown are each representative of two separate experiments.

FIG. 13 is a bar graph showing antisense p21^{Waf1/Cip1} oligodeoxynucleotide potentiates the cell cycle inhibitory (and presumably killing) effect of γ-irradiation on VSM cells exposed to serum, as described in Example III infra. The experiment shown is representative of two separate experiments.

FIGS. 14A and B illustrate how ionizing radiation inhibits DNA synthesis in VSM, but not A431, cells, as described in Example III, infra. Confluent (A) A10 VSM or (B) A431 cells were subjected to one of the following culture conditions: left in 10%-serum containing medium (continuous S); placed in serum-free medium the day of the experiment and left under those conditions for 48 h (continuous SF); placed in serum-free medium for 24 h and then stimulated with 10%-serum (SF→S) or PDGF-BB (30 ng/ml) (SF→PDGF-BB). All cells were irradiated with 8 Gy; when agonist was added, it was added 30 min after radiation. Six hours after agonist addition (where indicated), [³H]-thymidine (1 μCi/ml) was added to the medium overnight and DNA synthesis was assessed. Data is expressed as mean±s.e.m. of three wells per data point. * indicates p<0.05 compared to control (random sequence oligonucleotide). The experiments shown are representative of two separate experiments.

FIGS. 15A and B show the induction of p21 in VSM cells by ionizing radiation is blunted by antisense oligonucleotide to p21, as described in Example III, infra. Confluent VSM cells were transfected with antisense oligonucleotide to p21 or random sequence control oligonucleotide as described in Materials and Methods. (A) Non-transfected cells were exposed to ionizing radiation (12 Gy), lysed at the indicated times after exposure, and Western blotted with p21 antibody. (B) Cells transfected with the indicated oligonucleotides were treated similarly to (A). The arrowhead shows the band corresponding to p21. The experiments shown are representative of two separate experiments.

FIG. 16 depicts the induction of p21 in VSM cells by Adriamycin is blunted by antisense oligonucleotide to p21, as described in Example III, infra. The arrowhead shows the band corresponding to p21. The experiments shown are representative of two separate experiments.

FIG. 17 illustrates how the antisense oligonucleotide to p21 potentiates radiation-induced VSM cell cycle arrest, as described in Example III, infra. The experiment shown is representative of two separate experiments. *,#p<0.05 compared to control; +p<0.05 compared to random sequence oligonucleotide.

FIG. 18 demonstrates how the antisense oligonucleotide to p21 potentiates Adriamycin-induced VSM cell cycle arrest, as described in Example III, infra. The experiment shown is representative of two separate experiments. *,#p<0.05 compared to control; +p<0.05 compared to random sequence oligonucleotide.

FIG. 19A and B depict how Caspase-3 is activated by antisense oligonucleotide to p21 but not early after radiation or Adriamycin, as described in Example III, infra. Confluent VSM cells were transfected with oligonucleotide in the concentration indicated, left in serum-containing media overnight, and exposed to (A) ionizing radiation (12 Gy) or (B) Adriamycin where indicated. 4 h later, activation of caspase-3 was assessed by Western blotting. The arrowhead indicates the cleavage product of caspase-3 signifying its processing as an early step in apoptosis. Wortmannin (wort) is a positive control for apoptosis. The arrowhead shows the band corresponding to the cleavage product of caspase-3. The experiment shown is representative of two separate experiments.

FIGS. 20A-D show how the antisense oligonucleotide to p21 induces VSM cell apoptosis, as described in Example III, infra. VSM cells were grown on glass cover slips and transfected with (A,B) random sequence control oligonucleotide to p21 or (C,D) antisense oligonucleotide. 24 h later, the cells were fixed and stained in situ with Hoechst 33258. Representative microscopic fields were photographed under (A,C) visual or (B,D) UV light at 40×.

FIG. 21 shows that TGF-β decreases mitogenesis in serum-starved VSM cells, as described in Example IV, infra. * indicates significance difference from control (no TGF-β). The experiment shown is representative of three separate experiments.

FIG. 22 shows how TGF-β decreases 10% serum-stimulated mitogenesis in VSM cells cells, as described in Example IV, infra. * indicates significance difference from serum alone. The experiment shown is representative of two separate experiments.

FIG. 23 demonstrates the transfection of VSM cells with antisense p21 oligodeoxynucleotide specifically inhibits p21 protein level cells, as described in Example IV, infra. The experiment shown is representative of at least three separate experiments.

FIG. 24 illustrates that TGF-β remains inhibitory in VSM cells transfected with antisense p21 oligodeoxynucleotide cells, as described in Example IV, infra. A10 VSM cells were grown to confluence, transfected as described with either antisense (solid bars) or control (hatched bars) oligodeoxynucleotide, and serum-starved overnight. Subsequently, the cells were treated with 10% serum containing medium and/or TGF-β at the indicated concentrations (in ng/ml), and DNA synthesis assessed as in FIG. 22; absolute counts differ slightly from other experiments due to differences in starting confluency of the cells. * indicates significance difference from serum alone. The experiment shown is representative of two separate experiments.

FIG. 25 shows that the antisense p21 oligodeoxynucleotide decreases TGF-β-mediated laminin production and secretion cells, as described in Example IV, infra. The experiment shown is representative of three separate experiments.

FIG. 26 demonstrates how the antisense p21 oligodeoxynucleotide decreases TGF-β-mediated fibronectin production and secretion cells, as described in Example IV, infra. The experiment shown is representative of three separate experiments.

### DETAILED DESCRIPTION OF THE INVENTION

The present invention is based on the surprising discovery that p21^{Waf1/Cip1} protein serves a permissive role in PDGF-mediated cell growth and proliferation, such that its presence is required for the mitogenic effect of this growth factor, for example, in VSM cells. Therefore, successful therapy and prevention of abnormal growth and proliferation of cells must take into account p21^{Waf1/Cip1} activity or function to effectively combat proliferation of cells that result in disease states.

The methods and compositions of the invention can be used to treat a variety of diseases associated with abnormal cell growth and proliferation, including, but not limited to, atherosclerosis, angioplasty restenosis, renal mesangial cell proliferation and cancer, as well as preventing the VSM cell proliferation seen after coronary angioplasty, and may additionally be useful in cancer treatment as a sensitizer to chemotherapy and/or radiation (Mueller et al., 2000 Cancer Res. 2000. 60.(1):156.-63., 60, p. 156-163; and Wouters et al., 1997 Cancer Res., 57, p. 4703-4706). The methods may also be used to prevent plaques or tumors from forming.

The methods of the invention include regulation of cell growth mediated by CDKs by inhibiting p21^{Waf1/Cip1}, using a p21^{Waf1/Cip1} inhibitory agent to suppress abnormal cell growth and proliferation in VSM cells and other cells, including tumors (Mueller et al., 2000 Cancer Res. Jan. 1, 2000;60.(1):156.-63., 60, p. 156-163; and Wouters et al., 1997 Cancer Res., 57, p. 4703-4706).

Definitions

As used herein a “p21^{Waf1/Cip1}” and “p21” are used interchangeably.

Inhibition of cell growth and proliferation, as used herein, means an effective decrease in the number of cells treated with the compound of the invention e.g. antisense oligonucleotide of p21, as compared to non-treated cells.

As used herein a “p21^{Waf1/Cip1} inhibitory agent” is an agent that directly or indirectly inhibits activity of p21^{Waf1/Cip1}. A direct inhibitory agent, for example, is an antibody or antagonist that binds to and inhibits the activity of p21^{Waf1/Cip1}, soluble forms and fragments thereof having p21^{Waf1/Cip1}-binding activity, and new p21^{Waf1/Cip1} antagonists developed using well known methods for drug discovery as described herein and in the art. If the agent is p21^{Waf1/Cip1} specific (i.e. a direct inhibitory agent), it prevents proliferation of cells at the site of abnormal proliferation, such as the heart or the vascular system. An indirect inhibitor, such as an antisense oligonucleotide of p21^{Waf1/Cip1}, inhibits the synthesis or secretion of p21^{Waf1/Cip1}, by binding to the nucleic acid sequence of p21^{Waf1/Cip1} and/or inhibits the expression (i.e. transcription or translation) of p21^{Waf1/Cip1}, thereby reducing the amount of p21^{Waf1/Cip1} produced, or sequestering it away from its target protein.

Methods and Compositions of the Invention

The present invention provides methods and compositions to treat diseases associated with abnormal cell proliferation, by inhibiting the expression or activity of p21^{Waf1/Cip1}. In one embodiment, a p21^{Waf1/Cip1} inhibitory agent is administered to a subject at high risk for such diseases, for example atherosclerosis to prevent abnormal proliferation. Such high risk individuals can be prescreened using known medical procedures such as serum cholesterol measurements, history of premature heart disease, and invasive and non-invasive measurements of cardiac ischemia.

Included within the scope of p21^{Waf1/Cip1} indirect inhibitors of the invention are nucleic acids, including antisense oligonucleotides, that block the expression of p21^{Waf1/Cip1} genes within cells by binding a complementary messenger RNA (mRNA) and preventing its translation (Wagner, Nature 372:332-335 (1994); and Crooke and Lebleu, Antisense Research and Applications, CRC Press, Boca Raton (1993)). Gene inhibition may be measured by determining the degradation of the target RNA. Antisense DNA and RNA can be prepared by methods known in the art for synthesis of RNA including chemical synthesis such as solid phase phosphoramidite chemical synthesis or in vitro and in vivo transcription of DNA sequences encoding the antisense RNA molecule.

The antisense DNA sequences may be incorporated into vectors with RNA polymerase promoters such as the T7 or SP6 polymerase promoters. Alternatively, antisense cDNA constructs that synthesize antisense RNA constitutively or inducibly can be introduced into cell lines. The potency of antisense oligonucleotides for inhibiting p21^{Waf1/Cip1} may be enhanced using various methods including: 1) addition of polylysine (Leonetti et al., Bioconj. Biochem. 1:149-153 (1990)); 2) encapsulation into antibody targeted liposomes (Leonetti et al., Proc. Natl. Acad Sci. USA 87:2448-2451 (1990) and Zelphati et al., Antisense Research and Development 3:323-338 (1993)); 3) nanoparticles (Rajaonarivony et al., J. Pharmaceutical Sciences 82:912-917 (1993) and Haensler and Szoka, Bioconj. Chem. 4:372-379 (1993)), 4) the use of cationic acid liposomes (Felgner et al., Proc. Natl. Acad Sci. USA 84:7413-7417 (1987); Capaccioli et al., Biochem. Biophys. Res. Commun. 197:818-825 (1993); Boutorine and Kostina, Biochimie 75:35-41 (1993); Zhu et al., Science 261:209-211 (1993); Bennett et al., Molec. Pharmac. 41:1023-1033 (1992) and Wagner, Science 280:1510-1513 (1993)); 5) Sendai virus derived liposomes (Compagnon et al., Exper. Cell Res. 200:333-338 (1992) and Morishita et al., Proc. Natl. Acad. Sci. USA 90:8474-8478 (1993)), to deliver the oligonucleotides into cells: (6) the conjugation of the antisense oligonucleotides to a fusogenic peptide, e.g. derived from an influenza hemagglutinin envelope protein (Bongartz et al., Nucleic Acids Res. 22(22):4681-4688 (1994)).

Also included within the scope of p21 direct inhibitors of the invention are antagonists which bind to p21. The term “antagonists,” as it is used herein, refers to a molecule which, when bound to p21, decreases the amount or the duration of the effect of the biological activity of p21. Antagonists may include proteins, nucleic acids, carbohydrates, antibodies, or any other molecules which modulate the cell proliferation effects of p21. Suitable p21 antagonists can be readily determined using methods known in the art to screen candidate agent molecules for binding to p21, such as assays for detecting the ability of a candidate agent to measure CDK immunoprecipitation and check effect of immunopreciptated CDK on Rb phosphorylation (Sherr, C. J and Roberts J. M. Genes and Development, 13, 1501-1512 (1999)).

Direct inhibitors such as antibodies of the invention include polyclonal, monoclonal, chimeric, fragments, and humanized antibodies, that bind to p21 proteins or fragments of p21 proteins thereof. The most preferred antibodies will selectively bind to p21 proteins and will not bind (or will bind weakly) to non-p21 proteins. These antibodies can be from any source, e.g., rabbit, sheep, rat, dog, cat, pig, horse, mouse and human.

As will be understood by those skilled in the art, the regions or epitopes of a p21 protein to which an antibody is directed may vary with the intended application. For example, antibodies intended for use in an immunoassay for the detection of membrane-bound p21 on viable cells should be directed to an accessible epitope. The p21 proteins represents potential markers for screening, diagnosis, prognosis, and follow-up assays and imaging methods. In addition, based on the discoveries described herein, p21 proteins may be excellent targets for therapeutic methods such as targeted antibody therapy, immunotherapy, and gene therapy to treat conditions associated with the presence or absence of p21 proteins. Antibodies that recognize other epitopes may be useful for the identification of p21 within damaged or dying cells, for the detection of secreted p21 proteins or fragments thereof. Additionally, some of the antibodies of the invention may be internalizing antibodies, which internalize (e.g., enter) into the cell upon or after binding. Internalizing antibodies are useful for inhibiting cell growth and/or inducing cell death.

The invention includes a monoclonal antibody, the antigen-binding region of which competitively inhibits the immunospecific binding of any of the monoclonal antibodies of the invention to its target antigen. Further, the invention provides recombinant proteins comprising the antigen-binding region of any the anti-p21 monoclonal antibodies of the invention.

The invention also encompasses antibody fragments that specifically recognize a p21 protein or a fragment thereof. As used herein, an antibody fragment is defined as at least a portion of the variable region of the immunoglobulin molecule that binds to its target, i.e., the antigen binding region. Some of the constant region of the immunoglobulin may be included. Fragments of the monoclonal antibodies or the polyclonal antisera include Fab, F(ab′)₂, Fv fragments, single-chain antibodies, and fusion proteins which include the immunologically significant portion (i.e., a portion that recognizes and binds p21).

The chimeric antibodies of the invention are immunoglobulin molecules that comprise at least two antibody portions from different species, for example a human and non-human portion. Chimeric antibodies are useful, as they are less likely to be antigenic to a human subject than antibodies with non-human constant regions and variable regions. The antigen combining region (variable region) of a chimeric antibody can be derived from a non-human source (e.g. murine) and the constant region of the chimeric antibody, which confers biological effector function to the immunoglobulin, can be derived from a human source (Morrison et al., 1985 Proc. Natl. Acad. Sci. U.S.A. 81:6851; Takeda et al., 1985 Nature 314:452; Cabilly et al., U.S. Pat. No. 4,816,567; Boss et al., U.S. Pat. No. 4,816,397). The chimeric antibody may have the antigen binding specificity of the non-human antibody molecule and the effector function conferred by the human antibody molecule.

The chimeric antibodies of the present invention also comprise antibodies which are chimeric proteins, having several distinct antigen binding specificities (e.g. anti-TNP: Boulianne et al., 1984 Nature 312:643; and anti-tumor antigens: Sahagan et al., 1986 J. Immunol. 137:1066). The invention also provides chimeric proteins having different effector functions (Neuberger et al., 1984 Nature 312:604), immunoglobulin constant regions from another species and constant regions of another immunoglobulin chain (Sharon et al., 1984 Nature 309:364); Tan et al., 1985 J. Immunol. 135:3565-3567). Additional procedures for modifying antibody molecules and for producing chimeric antibody molecules using homologous recombination to target gene modification have been described (Fell et al., 1989 Proc. Natl. Acad. Sci. USA 86:8507-8511).

Humanized antibodies directed against p21 proteins are also useful. As used herein, a humanized p21 antibody is an immunoglobulin molecule which is capable of binding to a p21 protein. A humanized p21 antibody includes variable regions having substantially the amino acid sequence of a human immunoglobulin and the hyper-variable region having substantially the amino acid sequence of non-human immunoglobulin. Humanized antibodies can be made according to several methods known in the art (Teng et al., 1983 Proc. Natl. Acad. Sci. U.S.A. 80:7308-7312; Kozbor et al., 1983 Immunology Today 4:7279; Olsson et al., 1982 Meth. Enzymol. 92:3-16).

Various methods for the preparation of antibodies are well known in the art. For example, antibodies may be prepared by immunizing a suitable mammalian host with an immunogen such as an isolated p21 protein, peptide, fragment, or an immunoconjugated form of p21 protein (Harlow 1989, in: Antibodies, Cold Spring Harbor Press, NY). In addition, fusion proteins of p21 may also be used as immunogens, such as a p21 fused to -GST-, -human Ig, or His-tagged fusion proteins. Cells expressing or overexpressing p21 proteins may also be used for immunizations. Similarly, any cell engineered to express p21 proteins may be used. This strategy may result in the production of monoclonal antibodies with enhanced capacities for recognizing endogenous p21 proteins (Harlow and Lane, 1988, in: Antibodies: A Laboratory Manual. Cold Spring Harbor Press).

The amino acid sequence of p21 proteins, and fragments thereof, may be used to select specific regions of the p21 proteins for generating antibodies. For example, hydrophobicity and hydrophilicity analyses of the p21 amino acid sequence may be used to identify hydrophilic regions in the p21 protein structure. Regions of the p21 protein that show immunogenic structure, as well as other regions and domains, can readily be identified using various other methods known in the art (Rost, B., and Sander, C. 1994 Protein 19:55-72), such as Chou-Fasman, Garnier-Robson, Kyte-Doolittle, Eisenberg, Karplus-Schultz or Jameson-Wolf analysis. Fragments including these residues are particularly suited in generating anti-p21 antibodies.

Methods for preparing a protein for use as an immunogen and for preparing immunogenic conjugates of a protein with a carrier such as BSA, KLH, or other carrier proteins are well known in the art. Techniques for conjugating or joining therapeutic agents to antibodies are well known (Arnon et al., “Monoclonal Antibodies For Immunotargeting Of Drugs In Cancer Therapy”, in: Monoclonal Antibodies And Cancer Therapy, Reisfeld et al. (eds.), pp. 243-56 (Alan R. Liss, Inc. 1985); Hellstrom et al., “Antibodies For Drug Delivery”, in: Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in: Monoclonal Antibodies '84. Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982); Sodee et al., 1997, Clin. Nuc. Med. 21: 759-766). In some circumstances, direct conjugation using, for example, carbodiimide reagents may be used; in other instances linking reagents such as those supplied by Pierce Chemical Co., Rockford, Ill, may be effective.

Administration of a p21 immunogen is conducted generally by injection over a suitable time period and with use of a suitable adjuvant, as is generally understood in the art. During the immunization schedule, titers of antibodies can be taken to determine adequacy of antibody formation.

While the polyclonal antisera produced in this way may be satisfactory for some applications, for pharmaceutical compositions, monoclonal antibody preparations are preferred. Immortalized cell lines which secrete a desired monoclonal antibody may be prepared using the standard method of Kohler and Milstein (Nature 256: 495-497) or modifications which effect immortalization of lymphocytes or spleen cells, as is generally known. The immortalized cell lines secreting the desired antibodies are screened by immunoassay in which the antigen is the p21 protein or a fragment thereof. When the appropriate immortalized cell culture secreting the desired antibody is identified, the cells can be cultured either in vitro or by production in ascites fluid. The desired monoclonal antibodies are then recovered from the culture supernatant or from the ascites supernatant.

The antibodies or fragments may also be produced by recombinant means. The antibody regions that bind specifically to the desired regions of the p21 protein can also be produced in the context of chimeric or CDR grafted antibodies of multiple species origin.

The antibodies of the invention bind specifically to polypeptides having p21 sequences. In one embodiment, the p21 antibodies specifically bind to the extracellular domain of a p21 protein. In other embodiments, the antibodies of the invention specifically bind to other domains of a p21 protein or precursor, for example the antibodies bind to the cytoplasmic domain of p21 proteins.

Additionally, some of the antibodies of the invention are internalizing antibodies, i.e., the antibodies are internalized into the cell upon or after binding (Liu, H. et al., Cancer Res. 1998, 58, 4055-4060).

The methods of the invention comprise introducing the direct or indirect p21^{Waf1/Cip1} inhibitory agents so as to inhibit the activity of p21^{Waf1/Cip1}. For example, an anti-p21 mAb can be introduced into a subject to contact p21 positive cells to inhibit the activity of p21 and decrease the proliferation of cells.

In addition, the invention provides a process for the production of vaccines using p21 protein and a vaccine for treating cyclin-dependent kinase-mediated cell growth and proliferation. The vaccines contain a p21 protein, or partial sequences thereof, which is carrier-bound if desired, as an immunogen in a pharmacologically effective dose, and in a pharmaceutically acceptible formulation.

The production of these vaccines can be carried out according to known methods. However, the p21 proteins are preferably first lyophilized and subsequently suspended, if desired with addition of auxiliary substances.

Vaccination with these vaccines or combinations of vaccines according to the present invention can be carried out according to methods familiar to one skilled in the art (e.g. intradermally, intramuscularly, intraperitoneally, intravenously, subcutaneously or intranasally).

For intramuscular or subcutaneous administration, the vaccine can, for example, be suspended in physiological saline. For an intranasal or intraoccular application, the vaccine can, e.g., be used in the form of a spray or an aqueous solution. For a local, e.g. oral, administration, it is often necessary to temporarily protect the immunogens against inactivation, for example against proteolytic enzymes in the cavity of the mouth or in the stomach. Such temporary protection can be achieved by encapsulating the immunogens. This encapsulation can be carried out by coating with a protective agent (microencapsulation) or by embedding a multitude of immunogens according to the present invention in a protective carrier (macroencapsulation).

The encapsulation material can be semipermeable or become semipermeable when introduced into the human or animal body. A biologically degradable substance is usually used as a carrier for the encapsulation.

Administration of Inhibitors

The direct or indirect p21^{Waf1/Cip1} inhibitory agents may be administered to mammalian subjects, including: humans, monkeys, apes, dogs, cats, cows, horses, rabbits, mice and rats. The methods include administration by standard parenteral routes, such as subcutaneously, intravenously, intramuscularly, intracutaneously, intra-articularly, intrasynovially, intrathecally, periostally, or by oral routes. Alternative methods include, administration by implantable pump or continuous infusion, injection, or liposomes. Administration can be performed daily, weekly, monthly, every other month, quarterly or any other schedule of administration as a single dose injection or infusion, multiple dose, or in continuous dose form.

As is standard practice in the art, the direct or indirect p21^{Waf1/Cip1} inhibitory agents of the invention may be administered to the subject in any pharmaceutically acceptable carrier or adjuvant which is known to those of skill of the art. These carriers and adjuvants include, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances and polyethylene glycol.

The direct or indirect p21^{Waf1/Cip1} inhibitory agents may be administered to a subject in an amount and for a time sufficient to block the activity of p21^{Waf1/Cip1}, in the subject. The amount and time may also be sufficient to block p21^{Waf1/Cip1} positive cells direct or indirect p21^{Waf1/Cip1} inhibitory agents. The most effective mode of administration and dosage regimen for the inhibitors in the methods of the present invention depend on the severity of the abnormal proliferation of cells, the subject's health, previous medical history, age, weight, height, sex, response to treatment and the judgment of the treating physician. Therefore, the amount of inhibitors to be administered, as well as the number and timing of subsequent administrations are to be determined by a medical professional conducting therapy based on the response of the individual subject. Initially, such parameters are readily determined by skilled practitioners using appropriate testing in animal models for safety and efficacy, and in human subjects during clinical trials of candidate therapeutic inhibitor formulations. To determine if the amount administered is sufficient, the subject may be monitored for certain symptoms associated with the abnormal proliferation of cells.

Disruption of p53 (Bunz F, et al. J Clin Invest 104:263-269, 1999), and also of p21 (Wouters B G, et al. Cancer Res 57:4703-4706, 1997), sensitizes cancer cells to DNA damaging agents. Therefore, using the inhibitors of the invention in the methods of the invention, vascular cells may be rendered more sensitive to the effects of DNA damaging agents, such that targeted cells or tissues may be made more likely to become growth arrested and subsequently apoptotic, after p21 levels are attenuated. The invention also encompasses the use of the direct or indirect p21^{Waf1/Cip1} inhibitory agents of the invention together with other chemotherapeutic agents, such as adriamycin cisplatinum, carboplatin, vinblastine, vincristine, taxol, dactinomycin (actinomycin D), daunorubicin (daunomycin, rubidomycin), bleomycin, plicamycin (mithramycin), mitomycin (mitomycin C), methotrexate, cytarabine (AraC), azauridine, azaribine, fluorodeoxyuridine, deoxycoformycin, and mercaptopurine. In addition, those of skill in the art will appreciate that the compounds of the present invention can be used in conjunctive therapy with other known chemotherapeutic compounds.

The Examples, infra, include the demonstration that transfection of several lines of VSM cells with antisense oligodeoxynucleotide specific to p21^{Waf1/Cip1} correlated with decreased cyclin D1/cdk 4, but not cyclin E/cdk 2 association. The Examples also show a dose-dependent inhibition of PDGF-BB-stimulated DNA synthesis and cell proliferation. The Examples demonstrate that the presence of p21^{Waf1/Cip1} is required for growth factor-induced proliferation of VSM cells.

The following examples are presented to demonstrate the methods of the present invention and to assist one of ordinary skill in using the same. The examples are not intended in any way to otherwise limit the scope of the disclosure of the protection granted by Letters Patent granted hereon.

### EXAMPLE I

### p21^{Waf1/Cip1} Is Required For PDGF Induced Vascular Smooth Muscle Cell Proliferation

Materials: Human recombinant PDGF-BB was obtained from Upstate Biotechnology, Inc (UBI)(Lake Placid, N.Y.). Mouse monoclonal p21^{Waf1/Cip1} and p27^{Kip1} and cyclin D1, goat polyclonal cdk 2 and cdk 4, and rabbit polyclonal cyclin E antibodies were obtained from Santa Cruz Biotechnology (Santa Cruz, Calif.). Anti-goat horseradish peroxidase-conjugated IgG was obtained from BioRad (Richmond, Calif.), Lipofectin® was obtained form Life Technologies (Rockville, Md.). Reagents for the Enhanced Chemiluminescence system and [³H]thymidine were obtained from Amersham (Arlington Heights, Ill.). All other reagents, including mouse monoclonal α-actin antibody, were from Sigma (St. Louis, Mo.).

Cell culture, DNA synthesis, and proliferation assays: Cultures of both A10 and A7r5 rat aortic VSM cells were obtained from American Type Culture Collection (Rockville Md.). Bovine aortic smooth muscle cells were supplied by Martha O'Donnell (O'Donnell and Owen, 1986 Proc. Natl. Acad. Sci. U.S.A., 83, p. 6132-6136). All of the cell lines were maintained as described (Weiss et al., 1998 Am. J. Physiol., 274, p. C1521-C1529) and were used between passages 15 and 25. The cells were growth-arrested by placing them in serum-free quiescence medium, exposed to growth factors as indicated, and [³H]thymidine incorporation assessed as previously described (Weiss and Nuccitelli, 1992a J. Biol. Chem., 267, p. 5608-5613). Cell proliferation was assessed by counting of adherent cells on 4 representative fields under 100× magnification in each of 3 wells per experimental condition.

Antisense transfections: Phosphorothioate antisense oligodeoxynucleotides were synthesized by Oligonucleotides Etc. (Wilsonville, Oreg.). The p21^{Waf1/Cip1} antisense vector was designed around the start codon of rat p21^{Waf1/Cip1}, with sequence 5′-GAC ATC ACC AGG ATC GGA CAT-3′ (SEQ. ID NO.: 1). The sense p21^{Waf1/Cip1} sequence is 5′-ATG TCC GAT CCT GGT GAT GTC-3′ (SEQ. ID NO.: 2). The scrambled random sequence control oligodeoxynucleotide was 5′-TGG ATC CGA CAT GTC AGA-3′ (SEQ. ID NO.: 3). For the lipofection procedure, cells were grown to 90% confluence, the appropriate concentration of oligodeoxynucleotide was mixed with 6.6 μL of Lipofectin® per ml of Opti-MEM medium and was added to the cells for 4 h at 37° C. The cells were washed and serum-free medium (without oligodeoxynucleotide) was added overnight, the media was changed in the morning and the cells were incubated in serum-free medium for the times indicated.

Western blots: Cells were grown to confluence in 6 cm culture dishes and serum deprived. After transfection and or treatment with appropriate agonist, the cells were washed with phosphate-buffered saline and lysed in lysis buffer and the supernatant was Western blotted as described (Weiss et al., 1998 Am. J. Physiol., 274, p. C1521-C1529).

1. Determination of the Dependence of G1-Phase Progression on p21^{Waf1/Cip1} in the VSM Cell Lines.

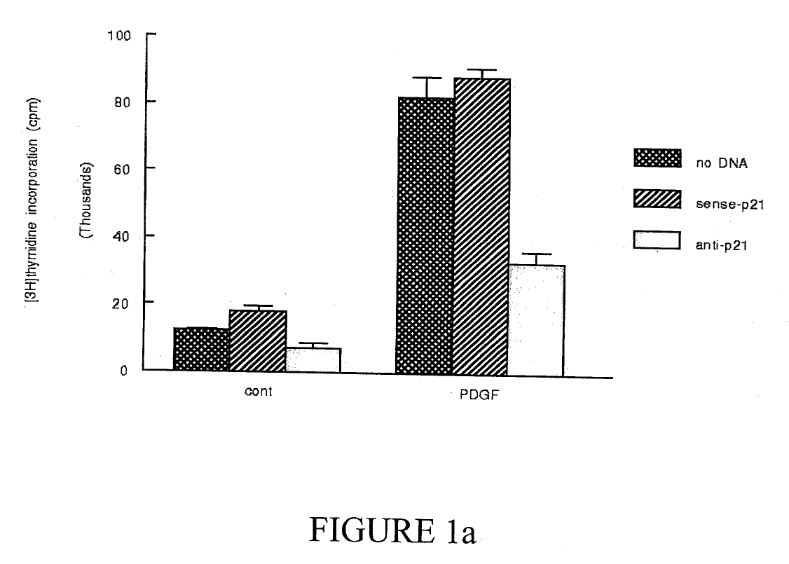

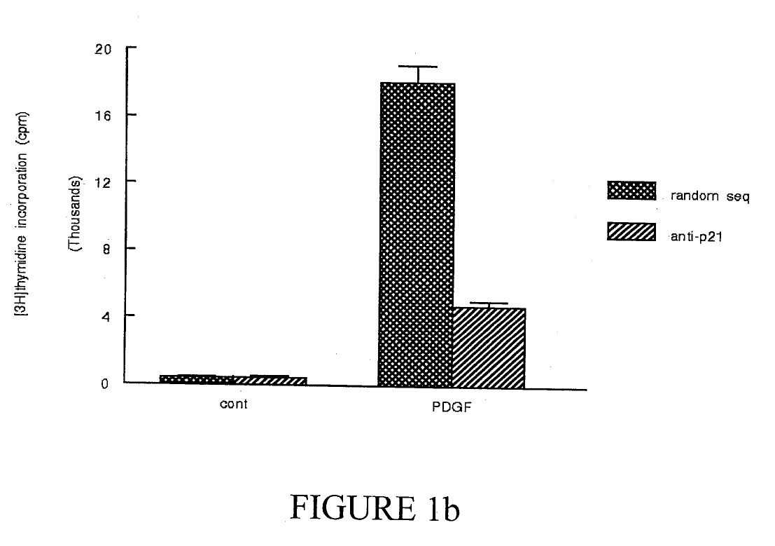

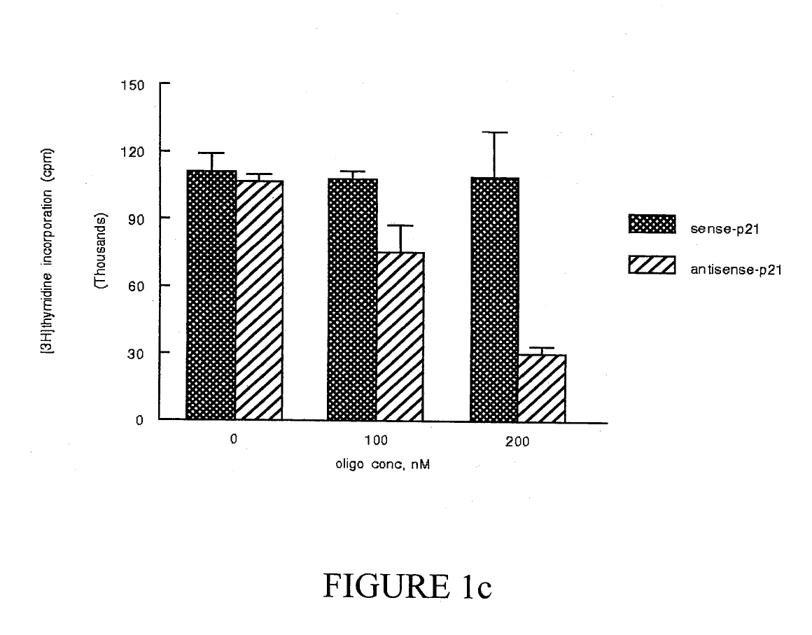

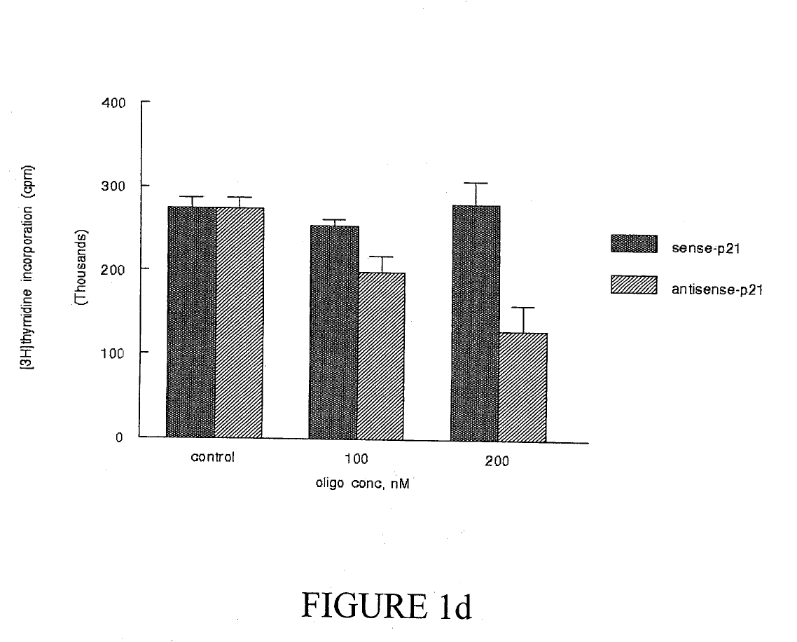

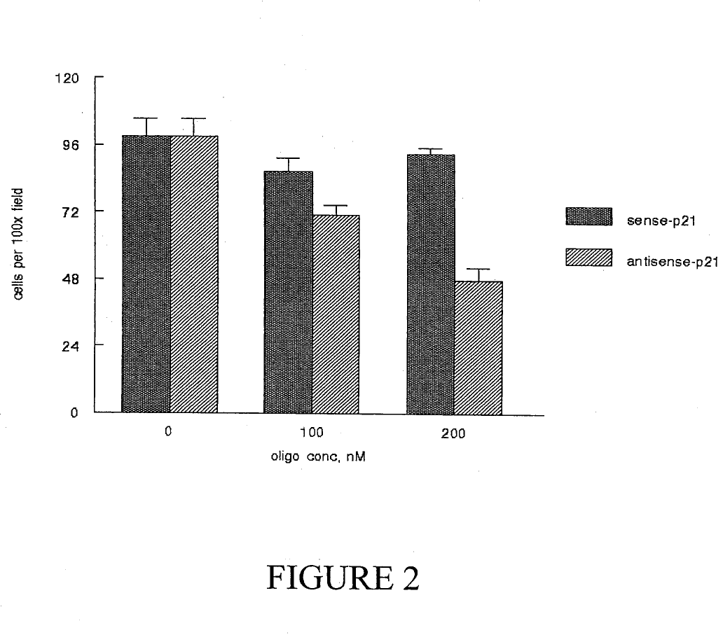

Antisense techniques were employed to examine the dependence of G1-phase progression on p21^{Waf1/Cip1} in the VSM cell lines (Crooke, 1993 Antisense research and applications. Boca Raton, CRC.). The oligodeoxynucleotides used were generated around the ATG start codon using GenBank sequences and were screened for lack of stable secondary structures or stable homodimer formation (OligoTech software, Oligonucleotides Etc., Wilsonville, Oreg.). Three independent controls were used in these experiments: (i) “dummy” transfection with Lipofectin but no DNA, (ii) random sequence oligodeoxynucleotide (SEQ. ID NO.: 3) transfection, and (iii) sense p21^{Waf1/Cip1} oligodeoxynucleotide (SEQ. ID NO.: 2) transfection. VSM cells were transfected with the appropriate oligodeoxynucleotide or control overnight in serum-free medium, and the next day the cells were stimulated with PDGF-BB (30 ng/ml) for another 18 h. DNA synthesis was assessed by [³H]-thymidine incorporation and is expressed as mean±s.e.m. of three wells per data point. The absolute counts differ between experiments due to different confluency of the cells. Significant inhibition of PDGF-stimulated DNA synthesis occurred when the cells were transfected with antisense p21^{Waf1/Cip1} (SEQ. ID NO.: 1), but not with sense p21^{Waf1/Cip1} (SEQ. ID NO.: 2), “dummy” transfection (FIG. 1a), or random sequence (SEQ. ID NO.: 3) control (FIG. 1b). To confirm that the observed growth inhibition was specific to the antisense p21^{Waf1/Cip1} oligodeoxynucleotide (SEQ. ID NO.: 1), dose/response analysis were performed. There was inhibition of DNA synthesis with increasing concentration of antisense p21^{Waf1/Cip1} oligodeoxynucleotide (SEQ. ID NO.: 1) up to 200 nM, with no effect of sense p21^{Waf1/Cip1} oligodeoxynucleotide (SEQ. ID NO.: 2) (FIG. 1c). To demonstrate that this effect was not specific to the A10 cell line, a similar effect in a bovine VSM cell line (FIG. 1d). A10 VSM cells were transfected as in FIG. 1c. After 18 h of PDGF incubation, the cells were counted by examining representative fields at 100× magnification. The average number of cells in 4 random fields in each well was determined. Changes in cell number were shown to parallel the alterations in DNA synthesis (FIG. 2).

2. Determination of Transfection Efficiency of the p21^{Waf1/Cip1} Antisense Oligonucleotide Agents.

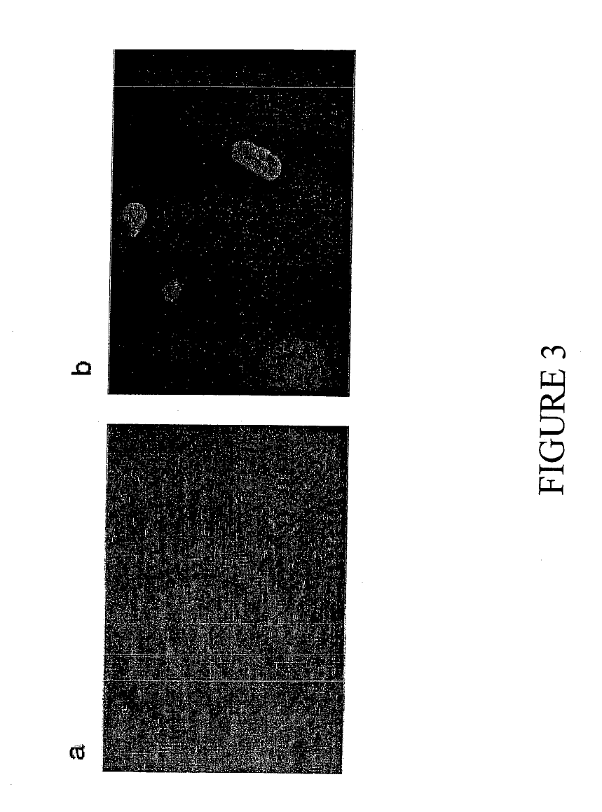

To establish whether the oligodeoxynucleotides crossed the cell membrane and entered the nucleus in order to inhibit p21^{Waf1/Cip1} protein production, cells were transfected with a fluorescein-tagged p21^{Waf1/Cip1} antisense oligodeoxynucleotide (with the same sequence as the p21^{Waf1/Cip1} antisense) and were examined for transfection efficiency. Upon examination by fluorescence microscopy, these cells demonstrated 100% transfection efficiency (FIG. 3), as has been reported for this technique (Coats et al., 1996 Science, 272, p. 877-880).

3. Determination of Effect of p21^{Waf1/Cip1} Antisense Oligonucleotides on p21^{Waf1/Cip1} Expression Levels.

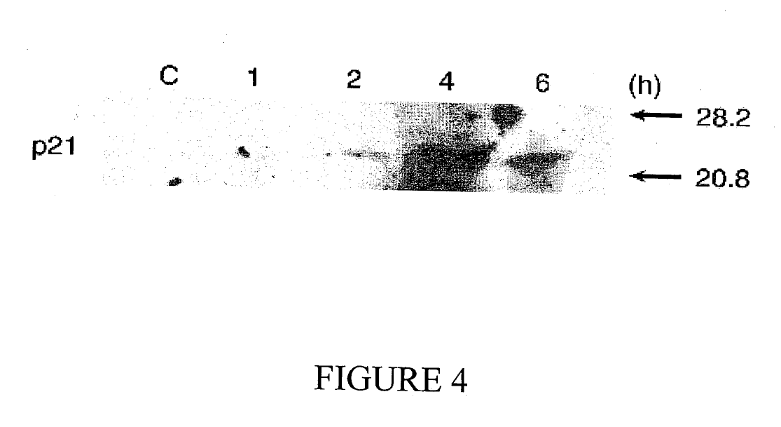

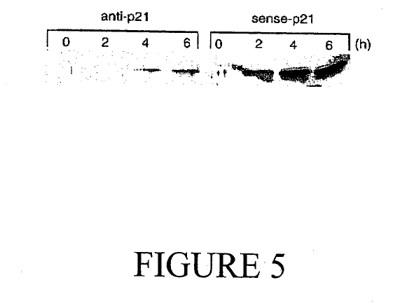

To determine whether antisense transfection with p21^{Waf1/Cip1} antisense oligonucleotides indeed decreases p21^{Waf1/Cip1} protein levels, p21^{Waf1/Cip1} levels after antisense transfection were examined employing the fact that PMA is a potent inducer of p21^{Waf1/Cip1} (Huang et al., 1995 Proc.Natl.Acad.Sci. U.S.A., 92, p. 4793-4797;Michieli et al., 1994 Cancer Res., 54, p. 3391-3395). Since p21^{Waf1/Cip1} protein levels were induced in VSM cells between 2 and 6 h after PMA stimulation (FIG. 4), the p21^{Waf1/Cip1} levels in transfected cells after similar times of PMA stimulation were examined. After transfection with appropriate oligodeoxynucleotide and subsequent overnight incubation in quiescent media, antisense p21^{Waf1/Cip1} oligodeoxynucleotide (SEQ. ID NO.: 1) caused significant attenuation of PMA-induced p21^{Waf1/Cip1} levels in VSM cells up to 6 h (FIG. 5). There was no effect of the p₂₁Waf1/Cip1 sense (SEQ. ID NO.: 2) control oligodeoxynucleotide on cellular p21^{Waf1/Cip1} levels (compare with FIG. 4).

4. Determination of Specificity of p21^{Waf1/Cip1} Antisense Oligonucleotides (SEQ. ID NO.: 1).

To check for specificity of protein inhibition by the antisense p21^{Waf1/Cip1} oligodeoxynucleotide (SEQ. ID NO.: 1), protein levels of p21^{Waf1/Cip1} and P27^{Kip1} after transfection with antisense p21^{Waf1/Cip1} oligodeoxynucleotide (SEQ. ID NO.: 1) were examined. In these experiments the ability of antisense oligodeoxynucleotides (SEQ. ID NO.: 1) to inhibit maximally stimulated CKI expression (see FIG. 4) was assessed. The cells were stimulated with PMA for 4h at various times after overnight serum starvation.

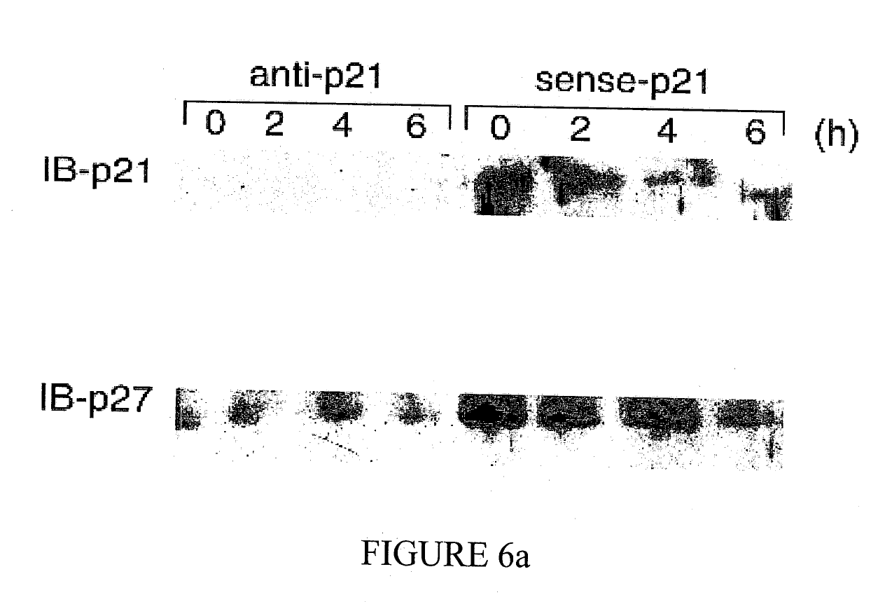

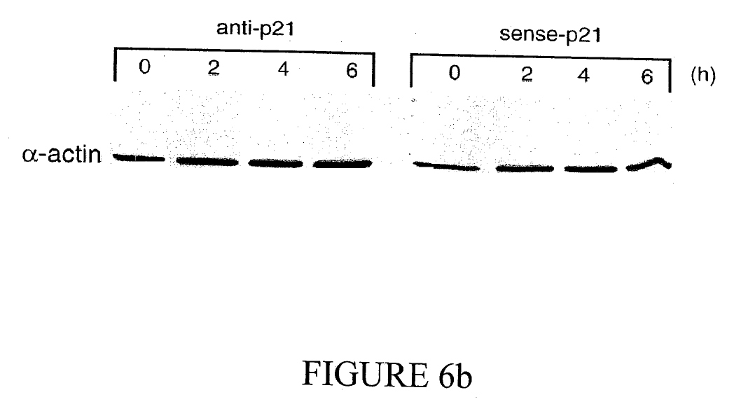

While antisense p21^{Waf1/Cip1} (SEQ. ID NO.: 1) completely inhibited p21^{Waf1/Cip1} protein even after maximal stimulation with PMA, there was a slight decrease in p27^{Kip1} protein as well with this oligodeoxynucleotide (FIG. 6a). At the times indicated after the overnight incubation (in hours), the medium was changed to quiescence medium and the cells were stimulated with PMA for 4 h to show maximal p21^{Waf1/Cip1} expression (FIG. 6). The lysates were immunoblotted with (a) p21^{Waf1/Cip1} or p27^{Kip1} antibody or (b) α-actin antibody. This is likely due to sequence similarity between the two genes, as p21^{Waf1/Cip1} shares 43% sequence identity with p27^{Kip1} in the cdk/cyclin binding site (residues 27-88), located in the conserved N terminus (Nomura et al., 1997 Gene, 191, p. 211-218; Toyoshima and Hunter, 1994 Cell, 78, p. 67-74).

Levels of the VSM cell structural protein α-actin were not altered after transfection under identical conditions (FIG. 6b), demonstrating that the effect of antisense oligonucleotides (SEQ. ID NO.: 1) on cell proteins was not a general inhibitory one. Furthermore, it is not believed that the slight p27^{Kip1} inhibition is playing a significant role in mitogenic inhibition, in light of data from other investigators (Rivard et al., 1996 J.Biol.Chem., 271, p. 18337-18341) based on the cyclin/cdk data discussed below.

5. Determination of the Effect of p21^{Waf1/Cip1} on the Association of Cyclin D1/cdk 4.

Cyclin D1/cdk 4 interaction was examined to determine this association as a possible mechanism of the permissive effect on growth of p21^{Waf1/Cip1} in VSM cells. Because other CKIs, such as p27^{Kip1}, have been shown to affect cyclin E/cdk 2 interaction (Cheng et al., 1998 Proc.Natl.Acad.Sci. U.S.A., 95, p. 1091-1096; Polyak et al., 1994 Cell, 78, p. 59-66), the nature of this association was also examined. While the CKIs have been shown to be growth inhibitors in VSM cells (Chang et al., 1995 J. Clin. Invest., 96, p. 2260-2268; Fukui et al., 1997 Atherosclerosis, 132, p. 53-59; and Matsushita et al., 1998 Hypertension, 31, p. 493-498), various CKIs have been reported to act as “assembly factors” in other cells, both in vivo (Cheng et al., 1999 EMBO J., 18, p. 1571-1583) and in vitro (LaBaer et al., 1997 Genes Dev., 11, p. 847-862), yet previous studies have not shown inhibition of growth with interference of cyclin/cdk association. Since the cyclin D1/cdk 4 interaction occurs early after growth factor stimulation (reviewed in (Arellano and Moreno, 1997 Int. J. Biochem. Cell Biol., 29, p. 559-573)) and because this interaction is facilitated by p21^{Waf1/Cip1} and p27^{Kip1} in vivo (LaBaer et al., 1997: Genes Dev., 11, p. 847-862).

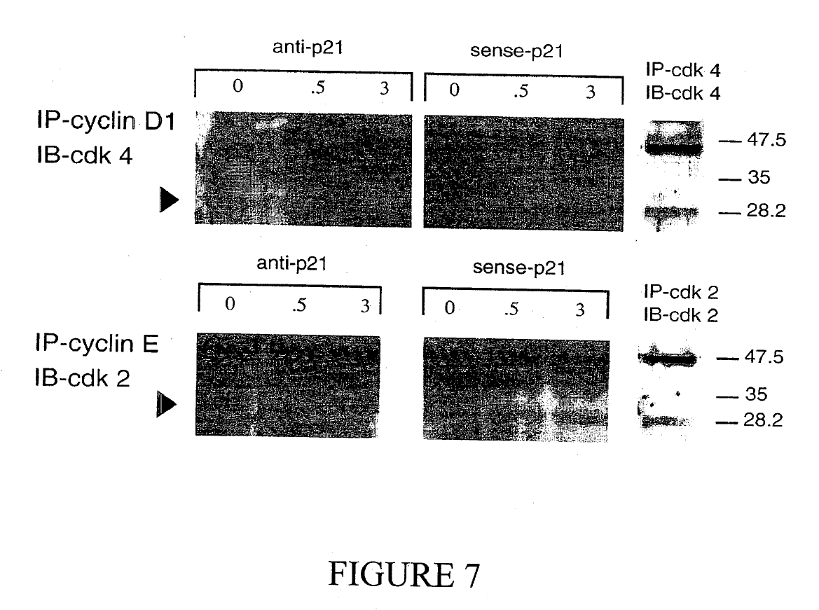

Cells were transfected with p21^{Waf1/Cip1} antisense (SEQ. ID NO.: 1) or sense oligodeoxynucleotide (SEQ. ID NO.: 2), allowed to grow overnight in serum-free media. After overnight incubation in serum-free medium, the cells were stimulated with PDGF-BB (30 ng/ml) for the times indicated. The cells were subsequently immunoprecipitated with either cyclin D1 or cyclin E and immunoblotted with cdk 4 or cdk 2, respectively. Antisense p21^{Waf1/Cip1}-transfected cells showed a marked decrease in association of cyclin D1 and cdk 4 at all times of PDGF stimulation, with no change in the cyclin E/cdk 2 interaction (FIG. 7). Thus the inhibitory effect of antisense p21^{Waf1/Cip1} oligodeoxynucleotide (SEQ. ID NO.: 1) in VSM cells is likely by means of disruption in cyclin D1/cdk 4 interaction and thus prevention of activation of cdk 4 by cyclin D1.

Abnormal proliferation of VSM-like cells is pathogenic for a variety of diseases, such as atherosclerosis and angioplasty restenosis (Ross, 1993 Nature, 362, p. 801-809), as well as renal mesangial cell proliferation (Megyesi et al., 1999 Proc. Natl. Acad. Sci. U.S.A., 96, p. 10830-10835), thus the mechanism by which these cells are stimulated to grow is important in designing antiproliferative therapies for treating these and other diseases. Published studies in VSM cells focus on the antiproliferative action of CKI overexpression (Chang et al., 1995 J. Clin. Invest., 96, p. 2260-2268; Fukui et al., 1997 Atherosclerosis, 132, p. 53-59; Matsushita et al., 1998 Hypertension, 31, p. 493-498; and Smith et al., 1997: Genes Dev., 11, p. 1674-1689), and there are even some studies promoting the idea that pharmacological methods to increase p21^{Waf1/Cip1} may be useful in preventing the VSM cell proliferation seen after coronary angioplasty (Kusama et al., 1999 Atherosclerosis, 143, p. 307-313; Takahashi et al., 1999 Circ.Res., 84, p. 543-550; and Yang et al., 1996 Semin. Interv. Cardiol., 1, p. 181-184).

The above results show for the first time that inhibition of p21^{Waf1/Cip1} efficiently blocks mitogen stimulated VSM cell proliferation.

The difference between data presented herein, as compared to that in the mouse cells, may well be due to cell type, but the finding of growth inhibition in cells lacking active p21^{Waf1/Cip1}, explains the “essential activator” role of p21 promulgated by that group (Cheng et al., 1999 EMBO J., 18, p. 1571-1583). Furthermore, since p21(−/−) mice appear to develop normally (Deng et al., 1995: Cell, 82, p. 675-684), it is conceivable that p21^{Waf1/Cip1} disruption only affects “adult” cells, or that redundant pathways for cell growth are not present in A10 cells. Nevertheless, the data herein is the first demonstrating that the presence of these pleiotropic molecules is required for growth-factor mediated G1 progression in any cell type.

While the antisense p21^{Waf1/Cip1} oligodeoxynucleotide (SEQ. ID NO.: 1) clearly inhibits p21, there is also slight inhibition of p27^{Kip1} protein as well by this oligodeoxynucleotide (FIG. 3). This occurrence is likely due to the sequence similarity between the two genes (Nomura et al., 1997 Gene, 191, p. 211-218; and Toyoshima and Hunter, 1994 Cell, 78, p. 67-74). However, it is not believed that the slight amount of inhibition of P27^{Kip1} is actively inhibiting VSM cell growth in the experiments, since cyclinE/cdk2 association is not affected (Coats et al., 1996 Science, 272, p. 877-880; Polyak et al., 1994 Cell, 78, p. 59-66; and Ravitz et al., 1995 Cancer Res., 55, p. 1413-1416) (see FIG. 7). Furthermore, others have reported suppression of quiescence, not of G1-phase progression, in fibroblasts transfected with antisense p27^{Kip1} (Rivard et al., 1996: J. Biol. Chem., 271, p. 18337-18341). In any case, the finding of unchanged α-actin protein levels in antisense p21 transfected cells argues against a general suppressive effect of these oligodeoxynucleotides (or of the process of transfection) on protein transcription.

Very recent work has shown that lack of a functional p21^{Waf1/Cip1} gene in transgenic mice ameliorates progression of chronic renal failure after partial renal ablation (Megyesi et al., 1999 Proc. Natl. Acad. Sci. U.S.A., 96, p. 10830-10835). PCNA was found to be significantly increased in p21(−/−) animals, but the degree of mesangial expansion was not quantitated. While the decrease in progression of renal failure was assumed to be due to a more hyperplastic (rather than hypertrophic) reaction in the p21(−/−) animals, the data disclosed in this example may shed some light on this phenomenon by suggesting that the response in p21(−/−) animals may have been a result of decreased mesangial cell mitogenesis due to inhibition of p21^{Waf1/Cip1} expression. This point of view is reinforced by others, noting that “all kidney growth parameters reported by Megyesi et al (Megyesi et al., 1999 Proc. Natl. Acad. Sci.U.S.A., 96, p. 10830-10835) are lower in p21(−/−) mice compared to p21(+/+) mice” (Al-Awqati and Preisig, 1999 Proc. Natl. Acad Sci. U.S.A., 96, p. 10551-10553).

Thus, these results support use of antisense p21^{Waf1/Cip1} oligonucleotides (SEQ. ID NO.: 1) to treat diseases involving abnormal VSM (or similar type) cell proliferation, such as atherosclerosis, angioplasty re-stenosis, and renal disease. While the available research on the CKIs in VSM cells has focused on the induction of the Cip/Kip family of CKIs in the presence of antiproliferative situations (Chen and Gardner, 1998 J. Clin. Invest., 102, p. 653-662; Fukui et al., 1997 Atherosclerosis, 132, p. 53-59; Kusama et al., 1999 Atherosclerosis, 143, p. 307-313; and Perlman et al., 1998 J. Biol. Chem., 273, p. 13713-13718), the inhibitory effects of CKI antisense constructs that were shown here may well be specific to vascular-like cells, as they were not observed in A431 cells (Example II, infra and reference (Ohtsubo et al., 1998 Oncogene, 16, p. 797-802).

### EXAMPLE II

### The Permissive Effect of p21^{Waf1/Cip1} on DNA Synthesis in p53-Inactive Cells

Materials: Human recombinant PDGF-BB was obtained from UBI (Lake Placid, N.Y.). Mouse monoclonal p21^{Waf1/Cip1} and cyclinD1, goat polyclonal cdk2 and cdk4, and rabbit polyclonal cyclinE antibodies were obtained from Santa Cruz Biotechnology (Santa Cruz, Calif.). Anti-goat horseradish peroxidase-conjugated IgG was obtained from Santa Cruz Biotechnology (Santa Cruz, Calif.). Lipofectin® was obtained form Life Technologies (Rockville, Md.). Reagents for the Enhanced Chemiluminescence system and [³H]thymidine were obtained from Amersham (Arlington Heights, Ill.). All other reagents, including mouse monoclonal α-actin antibody, were from Sigma (St. Louis, Mo.).

Cell culture and DNA synthesis: Cultures of A10 and A431 cells were obtained from American Type Culture Collection (Rockville Md.), were maintained as described (Weiss et al. 1998, Am. J. Physiol. 274, C1521-C1529), and were used between passages 15 and 24 or 25 and 35, respectively. The cells were growth-arrested by placing them in serum-free quiescence medium, exposed to growth factors as indicated, and [³H]thymidine incorporation assessed as previously described (Weiss and Nuccitelli, 1992, J. Biol. Chem. 267, 5608-5613).

Antisense transfections: Phosphorothioate antisense oligodeoxynucleotides were synthesized by Oligonucleotides Etc. (Wilsonville, Oreg.). The p21^{Waf1/Cip1} antisense vector was designed around the start codon of rat p21^{Waf1/Cip1}, with sequence 5′-GAC ATC ACC AGG ATC GGA CAT-3′ (SEQ. ID NO.: 1). The sense p21^{Waf1/Cip1} sequence is 5′-ATG TCC GAT CCT GGT GAT GTC-3′ (SEQ. ID NO.: 2). The scrambled random sequence control oligodeoxynucleotide was 5′-TGG ATC CGA CAT GTC AGA-3′ (SEQ. ID NO.: 3). For the lipofection procedure, cells were grown to 90% confluence, the appropriate concentration of oligodeoxynucleotide was mixed with 6.6 μL of Lipofectin® per ml of Opti-MEM medium and was added to the cells for 4 h at 37° C. Serum-free medium (without oligodeoxynucleotide) was added overnight, the media was changed in the morning and the cells were incubated in serum-free medium for the times indicated.

Western blots: Cells were grown to confluence in 6 cm culture dishes and serum deprived. After transfection and or treatment with appropriate agonist, the cells were washed with phosphate-buffered saline and lysed in lysis buffer and the supernatant was Western blotted as described (Weiss and Nuccitelli, 1992 J. Biol. Chem., 267, p. 5608-5613).

Example I shows that the CKI p21^{Waf1/Cip1}, while growth inhibitory in most situations, can also serve a permissive role in VSM cell growth. The mechanism behind this biphasic phenomenon is not yet known. In an attempt to further elucidate the nature of this effect, this study was designed to examine a cell line which is deficient in the immediate upstream regulator of p21^{Waf1/Cip1}. A431 cells, derived from a human squamous carcinoma, possess an inactive p53 protein (Kwok et al., 1994 Cancer Res., 54, p. 2834-2836) and are thus useful for assessing p53-independent effects of p21^{Waf1/Cip1}.

1. Determination of the Effect of p21^{Waf1/Cip1} in the A431 Cells.

In order to determine whether p21^{Waf1/Cip1} exerts a permissive effect on growth in cells which do not possess an active p53 protein, oligodeoxynucleotides encoding antisense (SEQ. ID NO.: 1) and sense (SEQ. ID NO.: 2) sequences which were generated around the ATG translational start codon were utilized as in Example I.

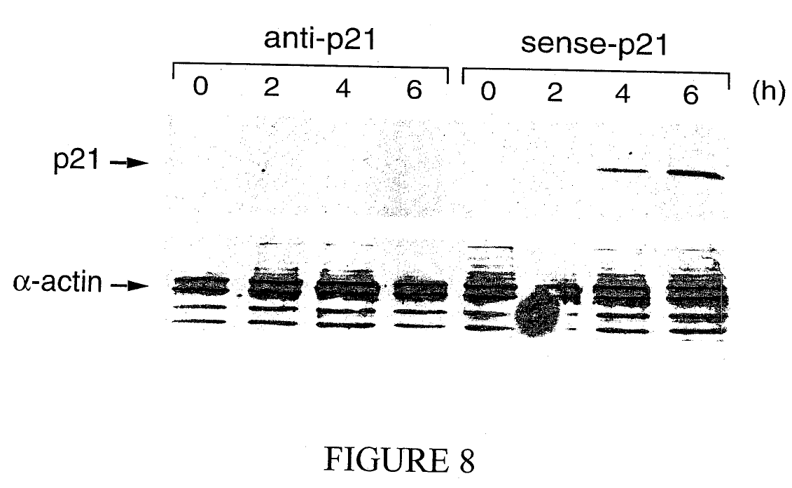

PMA is a potent inducer of p21^{Waf1/Cip1} in a variety of different cell lines (Huang et al., 1995 Proc.Natl.Acad.Sci. U.S.A., 92, p. 4793-4797; Michieli et al., 1994 Cancer Res., 54, p. 3391-3395) and was employed to examine p21^{Waf1/Cip1} levels after antisense transfection and confirm efficacy of translation to attenuate p21^{Waf1/Cip1} levels. A431 cells were transfected with 200 nM antisense p21^{Waf1/Cip1} (SEQ. ID NO.: 1) or sense p21^{Waf1/Cip1} oligodeoxynucleotide (SEQ. ID NO.: 2). After overnight incubation in serum-free medium, the cells were stimulated with PMA (100 ng/ml) for the times indicated, lysed, and the lysates were Western blotted with p21^{Waf1/Cip1} or α-actin antibody. Antisense p21^{Waf1/Cip1} oligodeoxynucleotide (SEQ. ID NO.: 1) caused significant attenuation of PMA-induced p21^{Waf1/Cip1} levels in A431 cells up to 6 h; there was no effect of the p21^{Waf1/Cip1} sense control oligodeoxynucleotide (SEQ. ID NO.: 2) on cellular p21^{Waf1/Cip1} levels as compared to other p53-independent cell lines (Zeng and el-Deiry, 1996, Oncogene 12, 1557-1564) when stimulated with phorbol ester (FIG. 8). Levels of the structural protein α-actin were not altered after transfection under identical conditions (FIG. 8), demonstrating that the effect of antisense oligonucleotides (SEQ. ID NO.: 1) on cell proteins was not a general inhibitory one towards protein translation.

2. Effect of p21^{Waf1/Cip1} as an “Assembly Factor” Role in Growth of A431 Cells

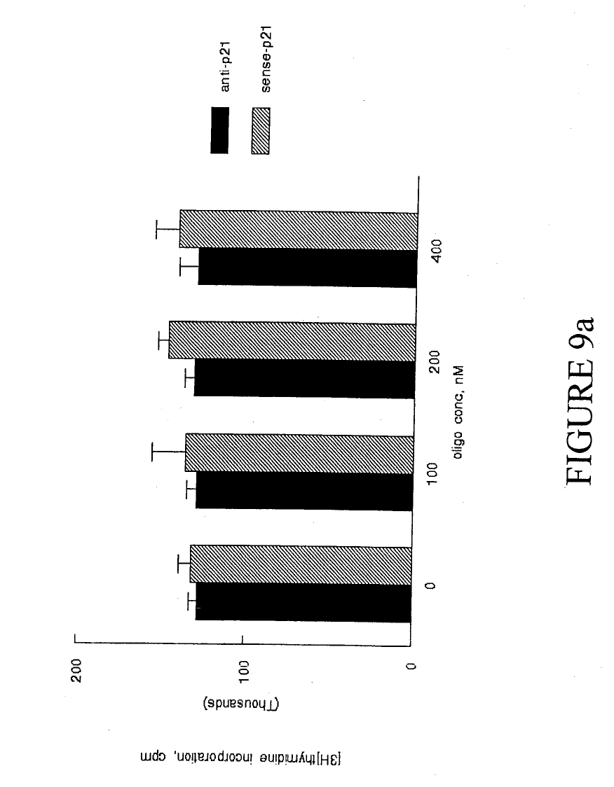

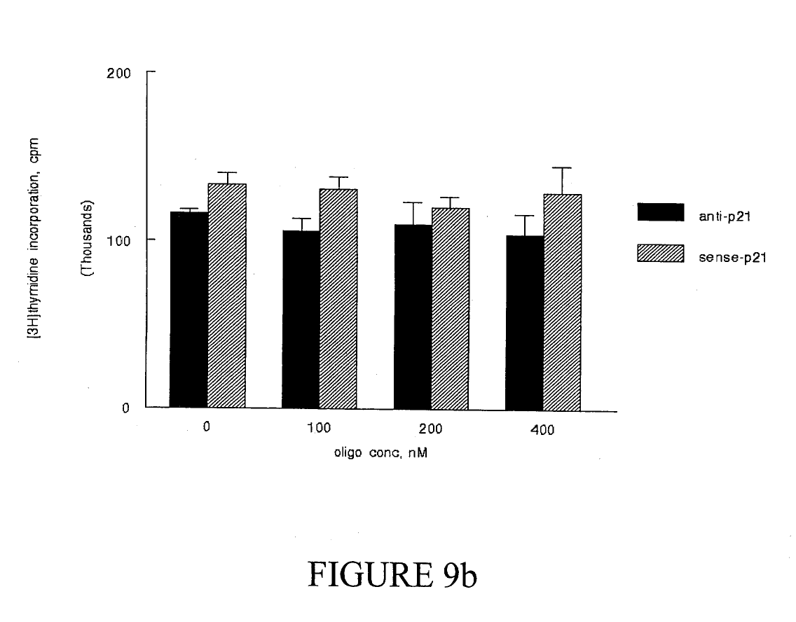

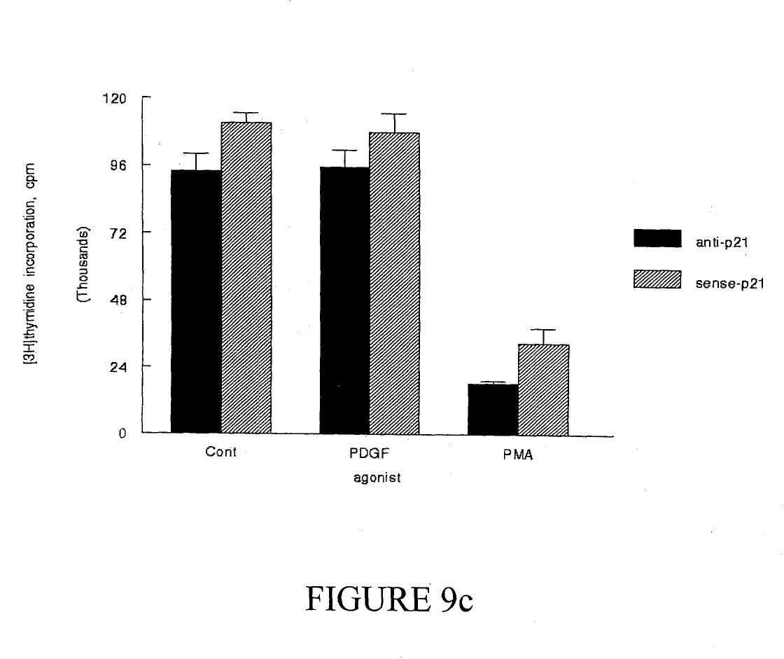

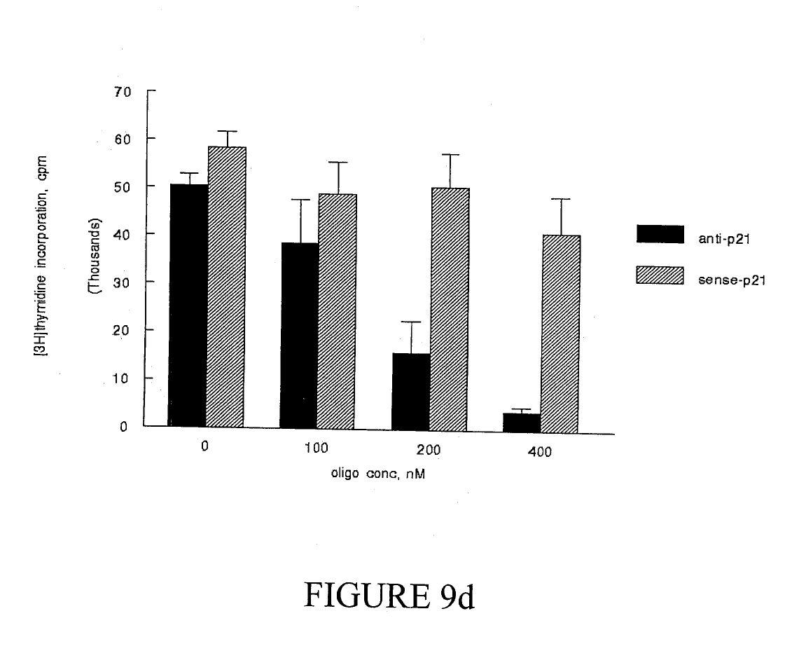

In order to determine whether p21^{Waf1/Cip1} serves an “assembly factor” role in growth of A431 cells as was observed in VSM cells of Example I, the DNA synthesis in these cells after transfection with antisense (SEQ. ID NO.: 1) or sense p21^{Waf1/Cip1} (SEQ. ID NO.: 2) oligodeoxynucleotides and subsequent stimulation with PDGF or serum was examined. There was no significant change in DNA synthesis upon stimulation of the cells with both of these growth factors (FIG. 9a,b), suggesting that p21^{Waf1/Cip1} does not serve an essential role in growth in A431 cells. Further experiments showed no difference in DNA synthesis between sense (SEQ. ID NO.: 2) and antisense oligodeoxynucleotide (SEQ. ID NO.: 1) up to 800 nM (data not shown). As a control for the ability of the transformed A431 cell line to in fact be growth inhibited, these cells were incubated with PMA and showed significant growth inhibition with this agent (FIG. 9c) as has been demonstrated in variety of other cell lines (Weiss et al., 1991 J. Cell. Physiol., 149, p. 307-312; and Weiss and Yabes, 1996 Am. J. Physiol. (Cell Physiol.), 270, p. C619-C627). This A431 data is in contrast with that obtained in a variety of VSM cell lines, where cell growth was inhibited by similar oligodeoxynucleotide concentrations in cell stimulated with both PDGF-BB and serum (FIG. 9d).

3. Determination of Mechanism of A431 vs VSM Cells Affected by Antisense p21^{Waf1/Cip1} (SEQ. ID NO.: 1).

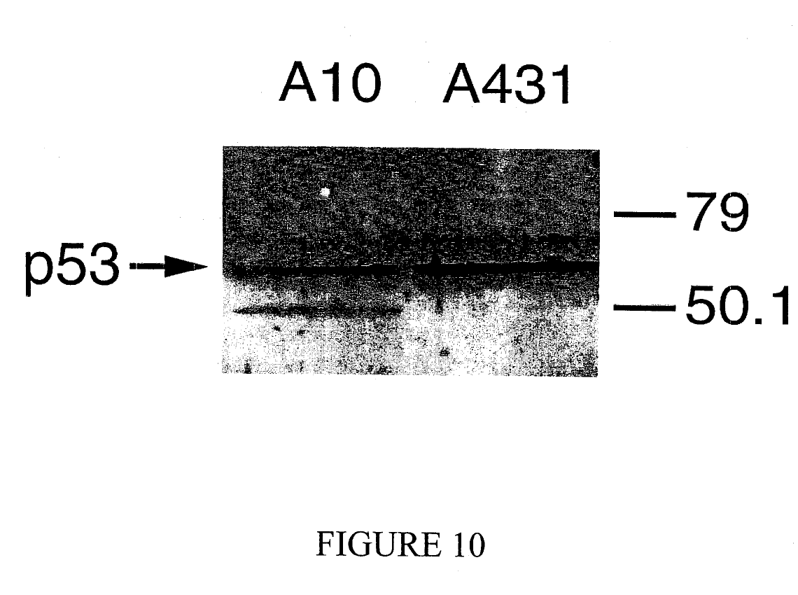

To begin to examine the mechanism by which A431 carcinoma cells are disparately affected by antisense p21^{Waf1/Cip1} (SEQ. ID NO.: 1) as compared to VSM cells, the Rb status of these cells compared with A10 VSM cells was first examined. It is known that A431 cells possess a mutant p53 protein which renders this protein inactive (Kwok et al., 1994 Cancer Res., 54, p. 2834-2836). Lysates from non-serum-starved A431 and A10 cells were Western blotted with p53 antibody which recognizes both wild type and mutant forms of p53. DNA damaging agents are able to up-regulate the mutant form of this protein in these cells (Kwok et al., 1994 Cancer Res., 54, p. 2834-2836), yet, despite the differences in p53 activity in the two cell types, similar levels of this protein are found in both lines (FIG. 10).

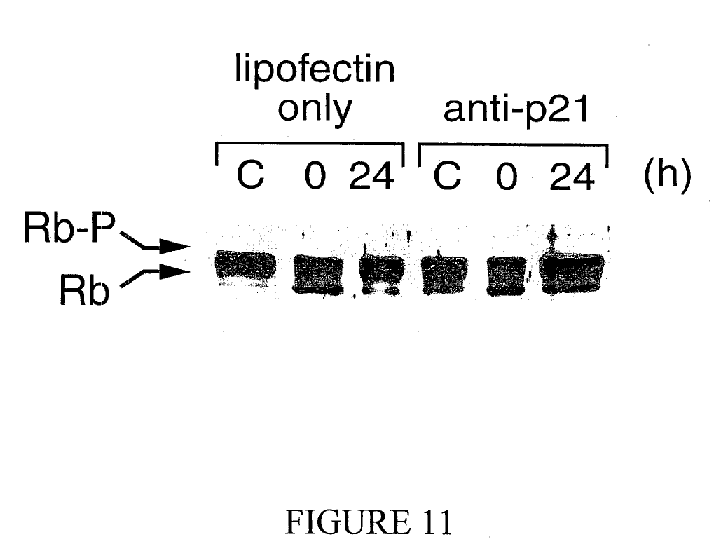

Upon activation by cyclin/cdk complexes, the Rb protein in turn becomes phosphorylated, causing it to release the transcription factors known as E2F, leading to early oncogene expression and ultimately to cell growth. An effect of p21^{Waf1/Cip1} on cdk/Rb interaction would be evident by a change in phosphorylation state of the Rb protein: this property can be assessed by examining small changes in gel mobility of this protein. A431 cells were transfected with 400 nM antisense p21^{Waf1/Cip1} oligodeoxynucleotide (SEQ. ID NO.: 1) or with lipofectin only (no DNA). After overnight incubation in serum-free medium, the cells were stimulated with complete media for 6 or 24 h, lysed, and the lysates were Western blotted with Rb antibody. Rb became hyperphosphorylated after 6 and 24 h of 10% serum stimulation as evidenced by the appearance of a higher molecular weight band, and there was no difference between cells transfected (as control) with no DNA and those transfected with antisense p21^{Waf1/Cip1} oligodeoxynucleotide (SEQ. ID NO.: 1) (FIG. 11), suggesting that p21^{Waf1/Cip1} has a minimal, if any, role in regulating this very distal part of the mitogenic signaling pathway in p53-inactive A431 cells.

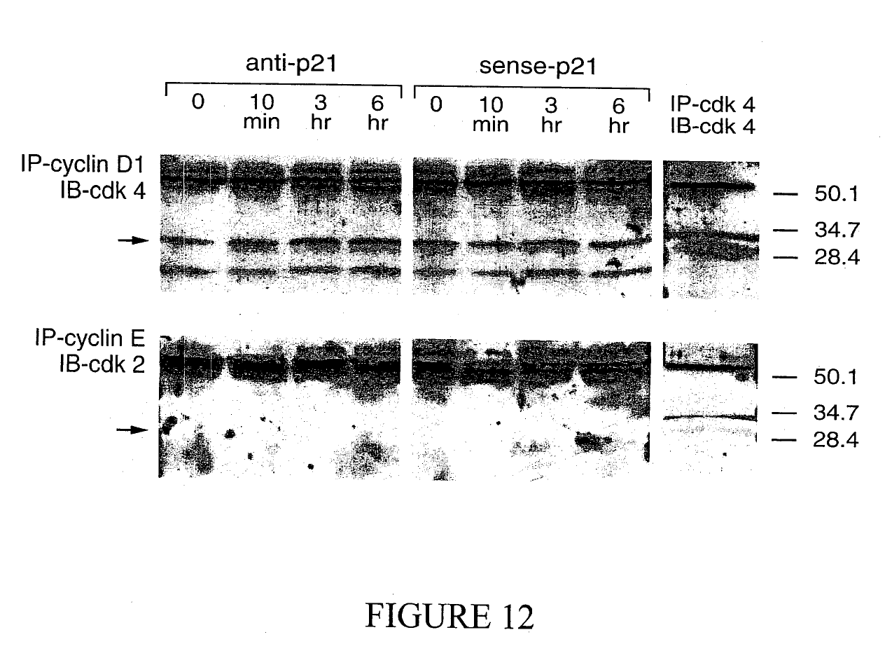

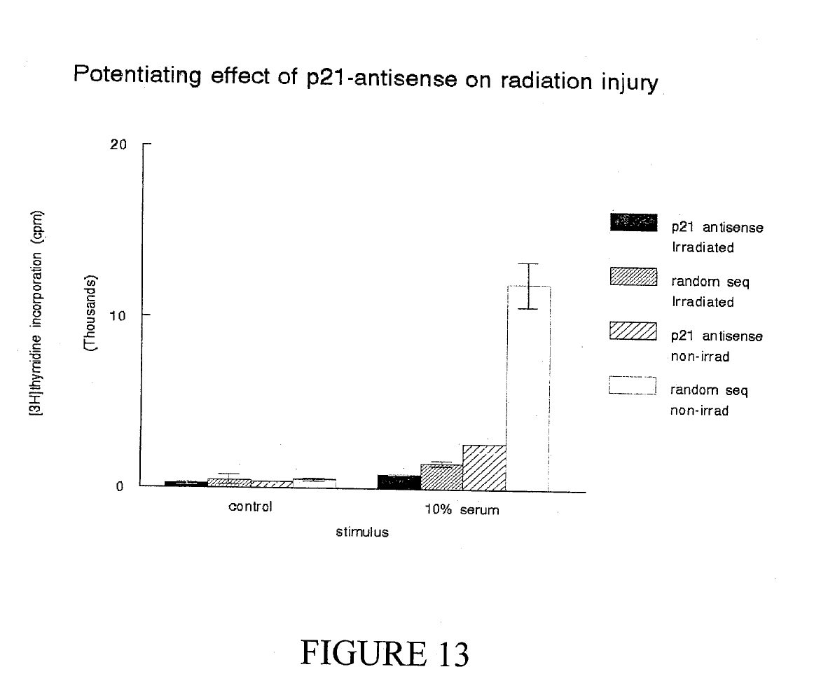

Cyclin/cdk interactions occur after growth factor stimulation and serve to integrate such responses with the CKIs and transmit them to the Rb/E2F systems, which leads to mitogenic signal transmission. The cyclin D1/cdk 4 interaction occurs early after growth factor stimulation (reviewed in (Arellano and Moreno, 1997 Int. J. Biochem. Cell Biol., 29, p. 559-573)), while the cyclinE/cdk2 interaction occurs late in G1 and is thought to have a role in triggering the actual onset of DNA replication after the cells have passed the restriction point (reviewed in (Sherr and Roberts, 1995: Genes Dev., 9, p. 1149-1163)). A431 cells were transfected with p21^{Waf1/Cip1} antisense (SEQ. ID NO.: 1) or sense SEQ. ID NO.: 2) oligodeoxynucleotide, allowed to grow overnight in serum-free media, and then stimulated for various times with PDGF-BB. The cells were subsequently immunoprecipitated with either cyclinD1 or cyclinE and immunoblotted with cdk4 or cdk2, respectively. A lysate sample, showing the mobility of the immunoprecipitated cdks, confirmed the identity of the cdks. A431 cells were transfected with antisense (SEQ. ID NO.: 1) or sense p21^{Waf1/Cip1} (SEQ. ID NO.: 2)oligonucleotides. After overnight incubation in serum-free medium, the cells were stimulated with PDGF-BB (30 ng/ml) for the times indicated and immunoprecipitated (IP) with cyclin D1 or cyclin E and immunoblotted (IB) with cdk4 or cdk2. Unlike the situation in A10 VSM cells of Example I, there was no change in cyclinD/cdk4 association when p21^{Waf1/Cip1} expression was inhibited by antisense oligodeoxynucleotide (SEQ. ID NO.: 1) (FIG. 12), suggesting that this protein is not required for association of these signaling proteins in these p53 inactive cells. Furthermore, there was no association of cyclinE with cdk2 at times ranging from 10 min to 6 h of PDGF exposure, and there was no effect of p21^{Waf1/Cip1} inhibition in these experiments (FIG. 12).

As shown above in Example I, that the CKI p21^{Waf1/Cip1} can play a permissive role in growth of VSM cells, acting by allowing assembly of the cyclinD1/cdk4 complex, which in turn leads to events resulting in cell cycle transit. In Example II, the focus was on the direct upstream influence on p21^{Waf1/Cip1}: the tumor suppressor p53. To determine if p53 has any influence on the nature of the p21^{Waf1/Cip1} effect (stimulatory versus inhibitory on cell growth) has not been examined.

Example II shows that the permissive effect of p21^{Waf1/Cip1} on cell growth is not universal, as it does not occur in A431 cells stimulated either with PDGF-BB or serum. While the cell lines used, A431 and A10, are two distinct cell types, one a squamous carcinoma line and the other a smooth muscle line, the growth factor-stimulated mitogenic signaling pathways are believed to be quite similar, with the most obvious difference being that the A431 cells lack a functional p53 protein because of a mutation in the gene encoding this protein (Kwok et al., 1994 Cancer Res., 54, p. 2834-2836). So the cells that display permissive effect of p21^{Waf1/Cip1} on cell growth are the optimal targets of the present invention.

While the most likely explanation of this data is that a functional p53 protein is necessary for the permissive effect of p21^{Waf1/Cip1}, there exist several alternative scenarios. While it was observed that p21^{Waf1/Cip1} protein expression is specifically decreased by the antisense p21^{Waf1/Cip1} oligodeoxynucleotide (SEQ. ID NO.: 1), it is conceivable that the magnitude of the decrement in A431 cells using antisense oligodeoxynucleotides was less than that seen with the VSM cells, or that A431 cells can signal to Rb with lower levels of p21^{Waf1/Cip1} protein.

While not wishing to be bound by any theory, since it has been well established (El-Deiry et al., 1993, Cell 75, 817-825) that p53 induces transcription of the p21^{Waf1/Cip1} gene (which would lead to an increase in p21^{Waf1/Cip1} protein levels), it is possible that there exist extremely low levels of p21^{Waf1/Cip1} in the absence of functional p53, as would occur in A431 cells. This may, in turn, result in the activation of alternative pathways which the cell has evolved to allow growth and circumvent any requirement for p21^{Waf1/Cip1} in cell cycle transit. That there exist alternate pathways for p53-mediated cell cycle arrest independent of p21^{Waf1/Cip1} is clear, since fibroblasts homozygous null for p21^{Waf1/Cip1} are only partially defective in their response to DNA damage (Brugarolas et al., 1995 Nature, 377, p. 552-557; Deng et al., 1995 Cell, 82, p. 675-684). This possible mechanism is further supported by the finding that primary fibroblasts from p21^{Waf1/Cip1} and p27^{Kip1}-null mice did not show overtly abnormal cell cycles, despite the finding by those investigators that overall cyclinD-dependent kinase activity was reduced below the assay limit of detectability (Cheng et al., 1999 EMBO J., 18, p. 1571-1583). Still other studies have shown an increased growth rate of p21(−/−) as compared to wild type mouse embryonic fibroblasts (Deng et al., 1995 Cell, 82, p. 675-684), and no apparent G1 block in human colorectal cancer cells (Waldman et al., 1995 Cancer Res., 55, p. 5187-5190). This is the first demonstration of the lack of a permissive role of p21^{Waf1/Cip1} in cells deficient in active p53.

Cells lacking p53 fail to arrest in response to a wide variety of DNA damaging agents. This has been shown to be due to the stabilization of the p53 protein and enhancement of its transcriptional activity leading to arrest at both G₂/M phases, possibly through transactivation of the 14-3-3 proteins (Hermeking et al., 1997 Mol. Cell, 1, p. 3-11), and at G₁/S, through up-regulation of p21^{Waf1/Cip1} (Brugarolas et al., 1995 Mol. Cell, 1, p. 3-11; and Deng et al., 1995: Cell, 82, p. 675-684). In light of this data, there may exist not only cross-talk between p53 and transcription of p21^{Waf1/Cip1}, but also an influence of the p53 protein on whether p21^{Waf1/Cip1} is growth inhibitory, or required for growth through an unknown mechanism.

In summary, the above Example II demonstrates that the permissive effect of the CKI p21^{Waf1/Cip1}, which was unequivocally demonstrated in A10 VSM cells, does not occur under similar conditions in A431 cells. Since the principle difference in the growth factor mitogenic signaling cascades between these two cell lines relates to a mutant and inactive p53 in the A431 cells, it is believed that the permissive effect of p21^{Waf1/Cip1} requires the presence of active p53 protein.

### EXAMPLE III

### Antisense p21^{Waf1/Cip1} Potentiates Ionizing Radiation- and Chemotherapy-induced Cell Cycle Arrest in VSM Cells

Materials: PDGF-BB and mouse monoclonal anti-human p21^{Waf1/Cip1} were obtained from Upstate Biotechnology (Lake Placid, N.Y.). Rabbit polyclonal anti-human caspase-3 antibody and anti-goat horseradish peroxidase-conjugated IgG were obtained from Santa Cruz Biotechnology (Santa Cruz, Calif.). Lipofectin® was obtained from Life Technologies (Rockville, Md.). Reagents for the Enhanced Chemiluminescence system and [³H]thymidine were obtained from Amersham (Arlington Heights, Ill.). Adriamycin (doxorubicin) was obtained from Pharmacia & Upjohn (Kalamazoo, Mich.). All other reagents, including Hoechst 33258, were from Sigma Chemical Co. (St. Louis, Mo.).

Cell culture and DNA synthesis assays: Cultures of A10 aortic VSM and A431 sarcoma cells were obtained from American Type Culture Collection (Rockville Md.), and were maintained as described (Weiss R H, et al. Am J Physiol 274:C1521-C1529, 1998); the A10 cells were used between passages 15 and 25. Where indicated, the cells were growth-arrested by placing them in serum-free quiescence medium, exposed to PDGF-BB or 10% serum-containing medium as indicated, and [³H]thymidine incorporation was assessed as previously described (Weiss R H, Nuccitelli R: J Biol Chem 267:5608-5613, 1992).

Ionizing radiation experiments: Cells were transfected with oligodeoxynucleotide 16-24 hours prior to γ-irradiation. Cells were subjected to 1-12 Gy of γ-irradiation from a ¹³⁷CS source. 30 minutes after irradiation cells were stimulated with PDGF-BB or 10% serum media. After 6 hours of stimulation, cells received 1 μCi [³H]thymidine per ml of media and were analyzed for DNA synthesis. For Western blots, cells were lysed 4 hours after irradiation unless stated otherwise.

Antisense transfections: Phosphorothioate antisense and random sequence control oligodeoxynucleotides were synthesized by Oligodeoxynucleotides Etc. (Wilsonville, Oreg.). The p21^{Waf1/Cip1} antisense vector was designed around the start codon of rat p21^{Waf1/Cip1}, with sequence 5′-GAC ATC ACC AGG ATC GGA CAT-3′ (SEQ. ID NO.: 1). The scrambled random sequence control oligodeoxynucleotide was 5′-TGG ATC CGA CAT GTC AGA-3′ (SEQ. ID NO.: 3). For the lipofection procedure, cells were grown to 60% confluence, washed with sterile phosphate-buffered saline, and the appropriate concentration of oligodeoxynucleotide was mixed with 6.6 μL of Lipofectin® per ml of Opti-MEM medium and was added to the cells for 4 h at 37° C. Serum-free medium (without oligodeoxynucleotide) was added overnight, the media was changed in the morning and the cells were stimulated as indicated.

Western blots: Cells were grown to confluence in 6 cm culture dishes and serum deprived. After transfection and or treatment with appropriate agonist, conditioned medium was removed and saved and the cells were washed with phosphate-buffered saline and lysed in lysis buffer. Both supernatant and cell lysate were normalized to the lysate protein concentrations and Western blotted as described (Weiss R H, et al. Am J Physiol 274:C1521-C1529, 1998).

Apoptosis assays: Cells were grown on collagen-coated coverslips, and transfected as stated above. 16-24 hours after transfection, cell were exposed to 12 Gy dose of γ-irradiation or 0.5 μM wortmannin. 24 hours later, cells were fixed in 3.7% formaldehyde (diluted in PBS) for 10 minutes. The cells were rinsed with cold PBS and permeabilized using 0.1% Triton X-100 (diluted in deionized water) for 5 minutes. Rinsed again with PBS, the cells on the coverslips were submerged in Hoechst Staining solution (3.0 μl in 37.5 ml deionized water) for 5 minutes. Cells were given a final three rinses with cold PBS before being mounted in polyvinyl alcohol mounting medium. Cell nuclei were visualized using a ZEISS WL Microscope and photographed under 40×.

1. Effect of Ionizing Radiation and Adriamycin on Growth of VSM Cells

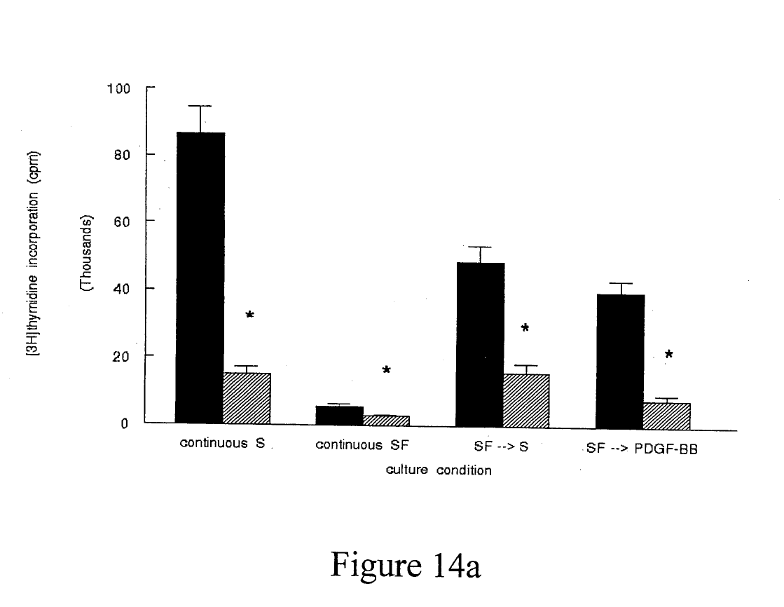

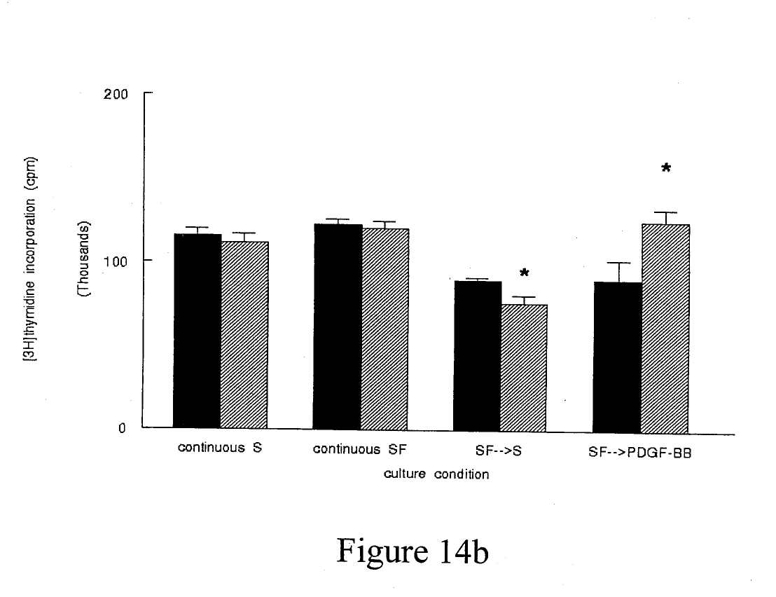

Both ionizing radiation and Adriamycin cause growth arrest due to induction of p53 as a result of DNA damage. This tumor suppressor protein sets in motion a series of events culminating in cell cycle arrest in G1 and G2 (Bunz F, et al. Science 282:1497-1501, 1998; Agarwal M L, et al. Proc Natl Acad Sci USA 92:8493-8497, 1995; and Poon R C, et al. J Biol Chem 271:13283-13291, 1996). Examples I and II utilized two established mesenchymal-derived cell lines, one (A10 VSM cells) possessing intact p53 and the other (A431 squamous carcinoma cells) which has a mutant p53 gene and an inactive p53 protein (Kwok T T, et al. Cancer Res 54:2834-2836, 1994). In order to characterize the response of these particular cells to DNA damage, growth of these cells in various stages of the cell cycle was examined. Upon removal of serum, “normal” cells generally remained in G0, and subsequent stimulation with serum or growth factors caused them to resume transit through the cell cycle. To set the stage for further studies, the cells were examined under four conditions: (1) when left in serum-containing medium; (2) when left in serum-free medium; (3) when stimulated with complete medium after serum-free medium, and (4) when stimulated with PDGF-BB after serum-free medium.

[³H]thymidine incorporation was examined, which is a measure of transit through S phase of the cell cycle, after the cells were exposed to irradiation. A10 VSM cells were growth arrested after exposure to ionizing radiation under all conditions examined, likely through p53 -mediated induction of p21 and subsequent cdk inhibition (FIG. 14A). In A431 cells, which possess a mutant and inactive p53, ionizing radiation failed to cause consistent inhibition of DNA synthesis, although there was slight (but significant) inhibition in cells which were exposed to serum after serum starvation (FIG. 14B). This suggested that p53, and its downstream effector p21 , are important in mediating the cell cycle arrest and DNA damage repair in cells exposed to ionizing radiation. Subsequent experiments in VSM cells were therefore performed in cells that were left in complete medium, since these were the conditions under which the changes with radiation were maximal, and because these conditions most closely replicate the in vivo milieu.

The above findings (FIG. 14A and B) suggest that p53 status is essential to determining the response of cells to ionizing radiation and, by extension, to DNA damage. This is consistent with data reported by other investigators, as the level of p53 has been shown to be a very sensitive indicator of DNA damage. It has been suggested that one double-stranded DNA break is sufficient to induce this protein (Di Leonardo A, et al. Genes Dev 8:2540-2551, 1994). Furthermore, both ionizing radiation and Adriamycin are known to cause DNA damage, and have been shown to increase p53 expression in order to mediate G1 and G2 arrest, such that DNA repair can occur (Bunz F, et al. Science 282:1497-1501, 1998; and Agarwal M L, et al. Proc Natl Acad Sci U S A 92:8493-8497, 1995). The downstream effector of p53 is p21, and since p21 has been shown to have variable effects on cell cycle events (Sherr C J, Roberts J M. Genes and Dev 13:1501-1512, 1999) and on growth (Weiss R H, et al. J Biol Chem 275:10285-10290, 2000; Weiss, R. H. and Randour, C. Cellular Signalling, 12:413-418, 2000), whether p21 attenuation affects cell growth in response to DNA damage, was studied.

2. Effect of p21^{Waf1/Cip1} Antisense Oligonucleotide (SEQ. ID NO.: 1) on Cell Growth in Response to DNA Damage

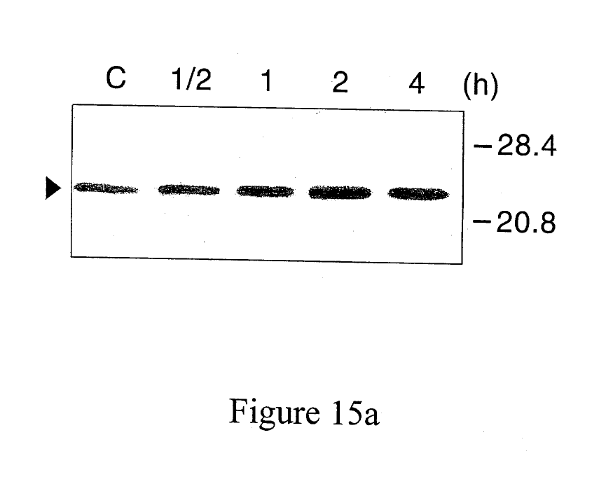

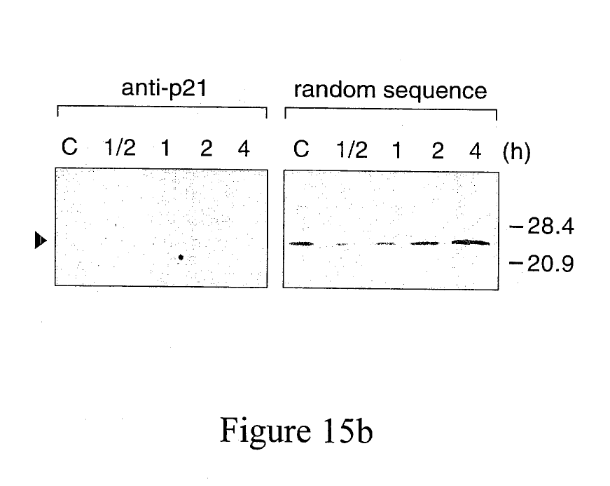

To confirm that p21 levels were increased after exposure to ionizing radiation, and that the oligonucleotides inhibit this phenomenon in A10 cells, p21 protein levels under these conditions were assayed. The level of p21 was increased after ionizing radiation (12 Gy) up to 4 h (FIG. 15A). p21 levels were consistently low in all cells transfected with the antisense oligonucleotide to p21 and then stimulated with ionizing radiation (12 Gy). No peak of induction was observed in these cells (FIG. 15B). In the cells transfected with the random sequence control oligonucleotide, there was initial slight inhibition of p21 levels at ½ and 1 h after ionizing radiation, but the levels rapidly increased by 4 h (FIG. 15b). With the exception of the initial decrease in p21 levels at ½ and 1 h, the results with the control oligonulceotides were similar to that observed in non-transfected cells. The specificity of the antisense oligonucleotide was previously demonstrated by showing no change in expression of α-actin in this cell line after antisense p21 oligonucleotide transfection (Weiss R H, et al. J Biol Chem 275:10285-10290, 2000; Weiss, R. H. and Randour, C. Cellular Signalling, 12:413-418, 2000).

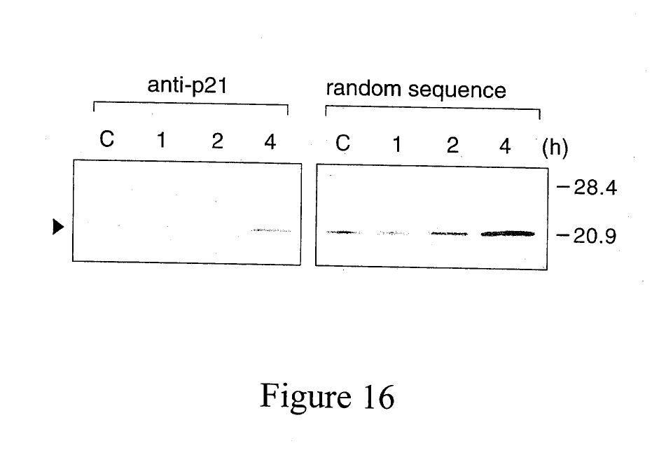

Adriamycin is a prototypical DNA damaging agent used in the treatment of a variety of cancers. The predominantly G2 arrest seen after Adriamycin treatment has been associated with an increase in p21 levels in some cell lines (Siu W Y, et al. FEBS Lett 461:299-305, 1999). In order to determine whether similar augmentation of p21 is seen in A10 VSM cells and whether the antisense p21 oligonucleotide is inhibiting this response, these cells were treated with Adriamycin either with or without first transfecting the cells with the antisense and control oligonucleotides. Confluent VSM cells were transfected with antisense oligonucleotide to p21 or random sequence control oligonucleotide as described above. After 24 h, the cells were exposed to Adriamycin (500 ng/ml), lysed at the indicated times after exposure, and Western blotted with p21 antibody. As in the case of cells exposed to ionizing radiation, p21 levels were increased by Adriamycin at similar times, with a marked attenuation of this response in cells transfected with the antisense p21 oligonucleotide(FIG. 16).

3. Effect of the Levels of p21 on Growth Activity

Having successfully inhibited the peak of p21 expression in VSM cells after ionizing radiation as well as Adriamycin exposure, it was determined whether growth modulation, after these maneuvers, is altered after suppression of the peak p21 level, which occurs in normal cells when so stimulated.

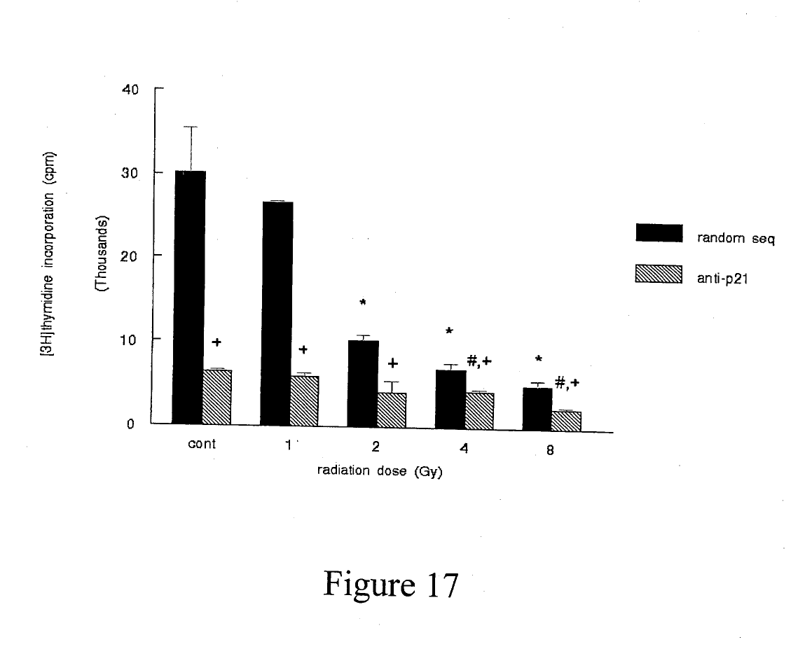

Confluent VSM cells were transfected with the antisense oligonucleotide to p21 (SEQ. ID NO.: 1) (or a random sequence control oligonucleotide(SEQ. ID NO.: 3) and exposed to various doses of ionizing radiation. After 24 h, the cells were exposed to the indicated dose of radiation. Six hours later, [³H]thymidine was added overnight and DNA synthesis was assessed as in FIG. 14. Cells transfected with the control oligonucleotide (SEQ. ID NO.: 3) showed attenuation of DNA synthesis as a function of radiation dose from 1 to 12 Gy (FIG. 17). After transfection of the cells with the antisense p21 oligonucleotide, there was a marked inhibition of DNA synthesis in non-irradiated cells, as previously shown in Example I (Weiss R H, et al. J Biol Chem 275:10285-10290, 2000). The effects of irradiation on cell cycle arrest were potentiated at higher doses of radiation, with a maximum potentiation at 8 Gy. The potentiation at 12 Gy did not reach statistical significance, probably due to cell mortality at that dose.

4. Effect of Antisense Oligonucletide-Mediated Inhibition of p21 on Adriamycin Induced Cell Cycle Arrest

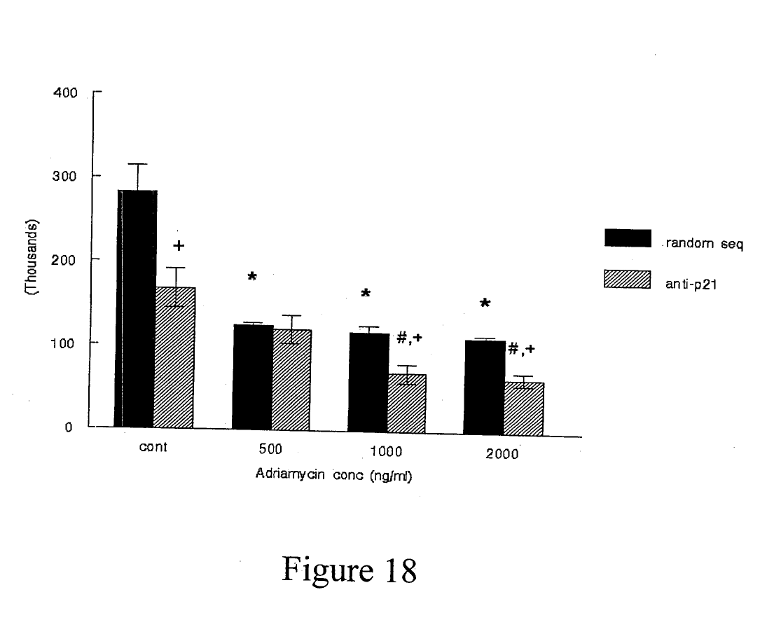

It has been previously shown that p21(−/−) cells are more sensitive to the killing effects of a variety of chemotherapeutic agents (Waldman T, et al. Nature 381:713-716, 1996; Stewart Z A, et al. Cancer Res 59:3831-3837, 1999; Waldman T, et al. Nat Med 3:1034-1036, 1997; and Fan S et al. Oncogene 14:2127-2136, 1997). The effect of antisense oligonucleotide-mediated inhibition of p21 on Adriamycin induced cell cycle arrest was determined. Confluent VSM cells were transfected with control (SEQ. ID NO.: 3) or antisense p21 oligonucleotides (SEQ. ID NO.: 1) and exposed to Adriamycin at concentrations from 500 to 2000 ng/ml. After 24 h, the cells were exposed for 2 h to the indicated concentration of Adriamycin. Two hours later, [³H]thymidine was added overnight and DNA synthesis was assessed as in FIG. 14. While the growth inhibitory effect of Adriamycin on control oligonucleotide transfected cells plateaued in this range, there was significant potentiation of growth inhibition in antisense p21 oligonucleotide transfected cells when exposed to from 1000 to 2000 ng/ml Adriamycin (FIG. 18).

5. Effect of Antisense Oligonucletide-Mediated Inhibition of p21 on Apotosis

While it was shown that p21 is required for serum and PDGF-stimulated growth in VSM cells (Weiss R H, et al. J Biol Chem 275:10285-10290, 2000), there are also reports that the absence of p21 may cause cells to be converted from growth arrest to apoptosis (Tian H, et al. Cancer Res 2000 Feb. 1;60 (3):679-84 60:679-684, 2000).

In order to determine whether the potentiation of ionizing radiation and Adriamycin induced DNA synthesis inhibition by p21antisense oligonucleotides is accompanied by apoptosis, caspase-3 activation in response to ionizing radiation was examined. Caspase-3 is an effector caspase whose activation (leading to apoptosis) results in a 20 kD cleavage product which can be assessed by Western blotting (McCarthy N J, Evan G I. Curr Top Dev Biol 36:259-278, 1998). This process is an early event in a cascade of reactions which ultimately leads to apoptosis, as is evident by the fact that inactivation of caspase-3 dramatically reduces apoptosis in several cell lines (Woo M, et al. Genes Dev 12:806-819, 1998).

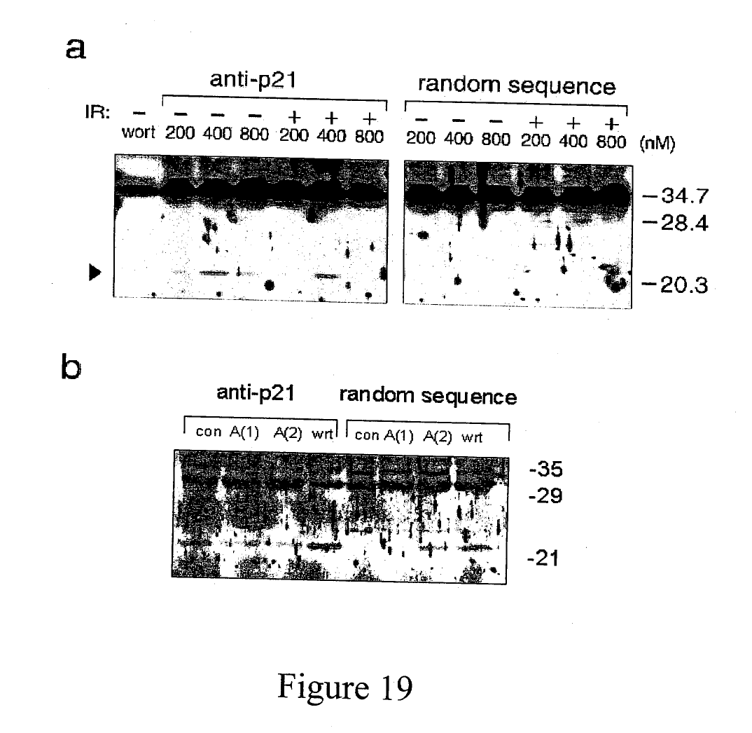

VSM cells were transfected with antisense or control oligonucleotides overnight and then stimulated with ionizing radiation. While ionizing radiation alone did not induce caspase-3 activation at 4 h at radiation doses of 12 Gy, there was an impressive increase in caspase-3 activation in cells transfected with antisense p21 oligonucleotide, either alone or after exposure to ionizing radiation (FIG. 19A).

Adriamycin increases p53 and p21 resulting in cell cycle arrest (Siu W Y, et al. FEBS Lett 461:299-305, 1999). As in the case of ionizing radiation, caspase-3 activation was observed in cells transfected with antisense p21 (SEQ. ID NO.: 1), but not random sequence oligonucleotides (SEQ. ID NO.: 3), with little effect of Adriamycin alone on this apoptosis effector at the time examined (FIG. 19b).

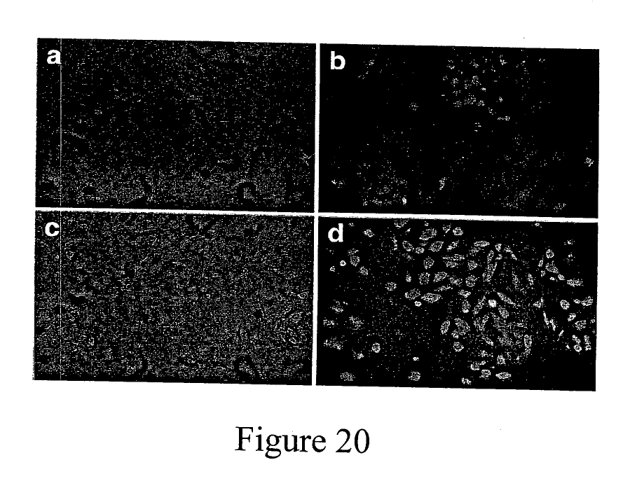

As another measure of apoptosis, transfected cells fixed and stained with Hoechst 33258 were examined. Cells transfected with antisense p21 (SEQ. ID NO.: 1), but not random sequence control oligonucleotides (SEQ. ID NO.: 3), showed extensive apoptotic changes 24 h after transfection (FIG. 20).

The results described in this Example show apoptosis, as well as potentiation of cell cycle arrest in VSM cells using a simple and straightforward technique, suggesting a new paradigm for treatment of fibrotic diseases, such as angioplasty restenosis, hemodialysis graft stenosis, mesangial proliferative glomerular disease, as well as in most pathogenic models of atherosclerosis. Indeed, the findings that transfection of the antisense p21 oligonucleotide alone induced apoptosis, coupled with the findings from other investigators that p21(−/−) mice do not have phenotypic changes or an increased susceptibility to spontaneous tumors (Deng C, et al. Cell 82:675-684, 1995), suggests the use of antisense p21 oligonucleotides as cancer therapeutics (see Tian H, et al. Cancer Res 2000 Feb. 1;60 (3):679-84 60:679-684, 2000).

While the CKIs had been considered to be solely growth inhibitory, data is emerging that these molecules in fact have both positive and negative effects on cell cycle checkpoints (Sherr C J, Roberts J M. Genes and Dev 13:1501-1512, 1999) as well as on cell growth (Weiss R H, et al. J Biol Chem 275:10285-10290, 2000). Furthermore, since p21 arrests transit through the cell cycle at G1 in order for DNA damage repair to occur (Bunz F, et al. Science 282:1497-1501, 1998), disruption of this checkpoint in p21(−/−) cells results in multiploidy and subsequent targeting of the cells for apoptosis (Waldman T, et al. Nature 381:713-716, 1996; and Mantel C, et al. Blood 93:1390-1398, 1999).

The above results demonstrate that using a novel antisense oligonucleotide corresponding to the translational start site of the p21 gene to attenuate p21 levels (Example I), arrest of the cell cycle and apoptosis occurs in VSM cells which have had p21 levels reduced using p21 inhibitory agents. Furthermore, under these conditions cell cycle inhibitory responses to radiation, and the DNA damaging chemotherapeutic agent, Adriamycin, are both potentiated.

While use of antisense oligonucleotides to p21 demonstrated a clear potentiation of apoptosis, after exposure of the cells to DNA damaging agents, it was surprising that caspase-3 activation did not occur with either radiation or Adriamycin alone at the times examined, despite inhibition of cell cycle transit as assessed by DNA synthesis. While not wishing to be bound by any particular theory, it is possible that caspase-3 was cleaved at a later time that was not apparent in the gels examined. In any case, the propensity of the antisense p21 oligonucleotide to initiate apoptosis and lead to its morphological characteristics in VSM cells is abundantly clear from these results.

The application of p21 inhibitors in renal disease, in addition to their role as vascular cell growth attenuators; has become evident in a recent study showing that p21(−/−) mice, as compared to wild type, were less likely to develop chronic renal failure after renal ablation (Megyesi J, et al. Proc Natl Acad Sci USA 96:10830-10835, 1999). While these investigators suggested that this effect was due to their finding that the absence of the p21 gene leads to a more hyperplastic response, apoptosis of mesangial or other renal cells may also be contributory. Other renal investigators have shown that diabetic p21(−/−) mice do not develop the same degree of glomerular hypertrophy as their wild type counterparts (Al Douahji M, et al. Kidney Int 56:1691-1699, 1999), an effect which may, in light of the data herein, also be due to apoptosis of glomerular cells.

The likelihood that antisense oligonucleotides may have potential therapeutic utility is further bolstered by experiments in animal models. Data has been generated on the pharmacology of antisense oligonucleotides in animal models. For example, the acute LD₅₀ of phosphorothioates is 500 mg/ml (Crooke, S. T. Therapeutic applications of oligonucleotides. 1995. Austin, R. G. Landes), well above the effects seen herein at nanomolar quantities of p21 antisense oligonucleotides. Furthermore, phosphorothioate oligonucleotides are rapidly and extensively absorbed after intravenous administration in rats, and distribute broadly to all peripheral tissues, especially liver, kidney, bone marrow, skeletal muscle and skin (Crooke, S. T. Therapeutic applications of oligonucleotides. 1995. Austin, R. G. Landes). An antisense oligonucleotide to c-raf injected into nude mice implanted with human tumors showed decent tissue uptake without the benefit of lipofection reagents (Monia B P, et al. J Biol Chem 267:19954-19962, 1992. The data presented herein showing the effect of the novel antisense cyclin kinase inhibitors of the invention in vascular cells support the use of the compositions and methods of the invention in the treatment of vascular and renal proliferative diseases.

### EXAMPLE IV

The Effect of p21^{Waf1/Cip1} on TBF-β-Mediated Matrix Protein Secretion

Materials: TGF-β1 and mouse monoclonal anti-human p21^{Waf1/Cip1} were obtained from Upstate Biotechnology (Lake Placid, N.Y.). Polyclonal anti-rat fibronectin and laminin antibodies were obtained from Chemicon (Temecula, Calif.). Anti-goat horseradish peroxidase-conjugated IgG was obtained from BioRad (Richmond, Calif.). Lipofectin® was obtained from Life Technologies (Rockville, Md). Reagents for the Enhanced Chemiluminescence system and [³H]thymidine were obtained from Amersham (Arlington Heights, Ill.). All other reagents, including mouse monoclonal α-actin antibody and protein A-Sepharose beads, were from Sigma Chemical Co.(St. Louis, Mo.).

Cell culture, DNA synthesis, and proliferation assays: Cultures of A10 aortic VSM cells were obtained from American Type Culture Collection (Rockville Md), were maintained as described (Weiss, R. H.; et al. Am.J.Physiol. 274: C1521-C1529; 1998), and were used between passages 15 and 25. The cells were growth-arrested by placing them in serum-free quiescence medium, exposed to TGF-β or 10% serum-containing medium as indicated in the figures, and [³H]thymidine incorporation was assessed as previously described (Weiss, R. H.; Nuccitelli, R. J. Biol. Chem. 267: 5608-5613; 1992).

Oligodeoxynucleotide transfections: Phosphorothioate antisense and random sequence control oligodeoxynucleotides were synthesized by Oligonucleotides Etc. (Wilsonville, Oreg.). The p21^{Waf1/Cip1} antisense vector was designed around the start codon of rat p21^{Waf1/Cip1}, with sequence 5′-GAC ATC ACC AGG ATC GGA CAT-3′ (SEQ. ID NO.: 1). The scrambled random sequence control oligodeoxynucleotide was 5′-TGG ATC CGA CAT GTC AGA-3′ (SEQ. ID NO.: 3). For the lipofection procedure, cells were grown to 60% confluence, washed with sterile phosphate-buffered saline, and the appropriate concentration of oligodeoxynucleotide was mixed with 6.6 μL of Lipofectin® per ml of Opti-MEM medium and was added to the cells for 4 h at 37° C. Serum-free medium (without oligodeoxynucleotide) was added overnight, the media was changed in the morning, and the cells were stimulated with TGF-β or serum as indicated.

Immunoprecipitations: VSM cells were grown to confluence. After incubation under appropriate conditions, the cells were washed with ice-cold phosphate-buffered saline and immediately lysed in lysis buffer (20 mM Tris [pH7.5], 150 mM NaCl, 1 mM EDTA, 1 mM EGTA, 1% Triton X-100, 2.5 mM sodium pyrophosphate, 1 mM β-glycerolphosphate, 1 mM Na₃VO₄, 1 μg/ml Leupeptin, 1 mM PMSF) at 4° C. The cells were scraped off with a rubber spatula and the insoluble material removed by centrifuging at 10,000×g for 10 min at 4°0 C. Protein concentration was determined by A₅₉₅, and lysates containing equal amounts of protein were incubated with 4 ml anti-fibronectin antibody at 4° C. overnight. Protein A-Sepharose beads were added and the resulting mixture was incubated for an additional 2 h at 4° C. The beads were centrifuged in a microfuge for 20 sec, and the pellet was washed 3 times with cold lysis buffer. The supernatant was decanted, gel loading buffer was added to the precipitate, and the solution was boiled for 5 min, and centrifuged. The supernatant was electrophoresed on a 7.5% SDS-polyacrylamide gel with equal volumes of sample per lane. The proteins were electrophoretically transferred to nitrocellulose and probed with fibronectin antibody.

Western blots: Cells were grown to confluence in 6 cm culture dishes and serum deprived. After transfection and/or treatment with appropriate agonist, conditioned medium was removed and saved and the cells were washed with phosphate-buffered saline and lysed in lysis buffer. Both supernatant and cell lysate were normalized to the lysate protein concentrations and Western blotted as described (Weiss, R. H.; et al. Am.J.Physiol. 274: C1521-C1529; 1998).

Results

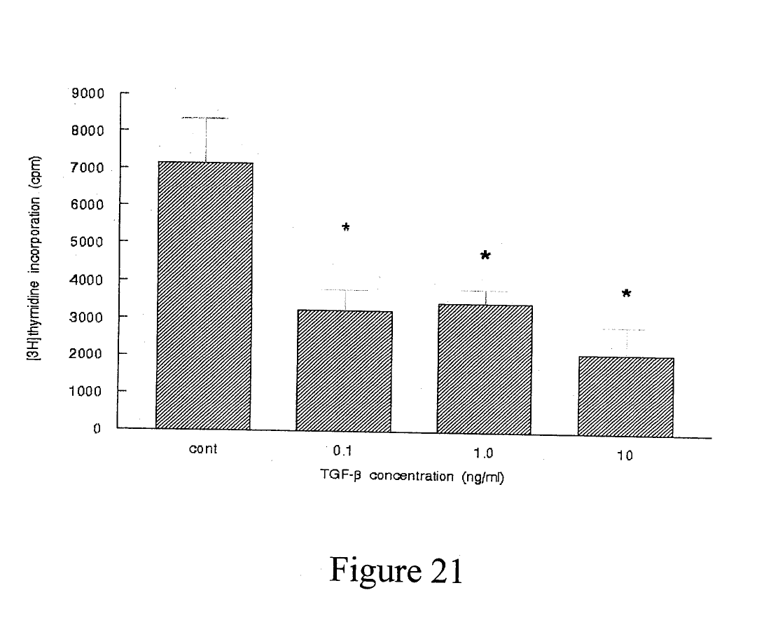

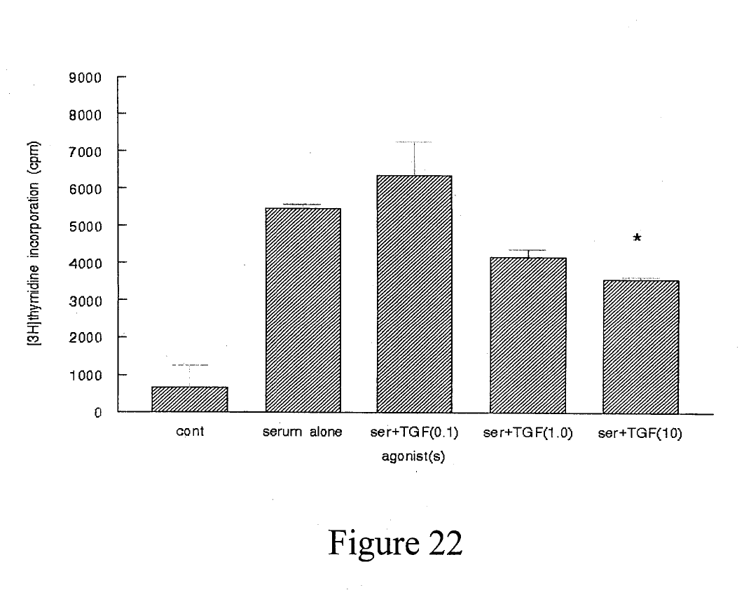

TGF-β has a bimodal effect on mitogenesis, being stimulatory or inhibitory depending on cell confluency and cell type (Moses, H. L.; et al. Cell 63: 245-247; 1990; and Centrella, M. et al. J. Biol. Chem. 262: 2869-2874; 1987). However, TGF-β is largely growth inhibitory in vivo (reviewed in Moses, H. L.; et al. Cell 63: 245-247; 1990). This property was confirmed in early passage rat VSM cells (Weiss, R. H.; et al. Kidney Int. 48: 738-744; 1995) and late passage rat mesangial cells (Weiss, R. H.; Ramirez, A. Nephrol. Dial. Transplant. 13: 2804-2813; 1998). To determine whether A10 VSM cells behave similarly, these cells were serum-starved for 24 h prior to stimulation with from 0.1 to 10 ng/ml TGF-β, and their ability to incorporate [³H]thymidine into DNA was examined (expressed as mean±s.e.m. of three wells per data point). At all concentrations tested, DNA synthesis was significantly reduced with the addition of TGF-β for 24 h (FIG. 21). To determine whether this growth inhibitory effect persisted when the A10 VSM cells were stimulated to enter G₁ with the addition of serum, the effect of the addition of 10% serum-containing media on TGF-β-exposed cells at the same concentrations was examined and found to exhibit similar growth-inhibitory results (FIG. 22). DNA synthesis was assessed by [³H]-thymidine incorporation and is expressed as mean±s.e.m. of three wells per data point; absolute counts differ slightly from other experiments due to differences in starting confluency of the cells.

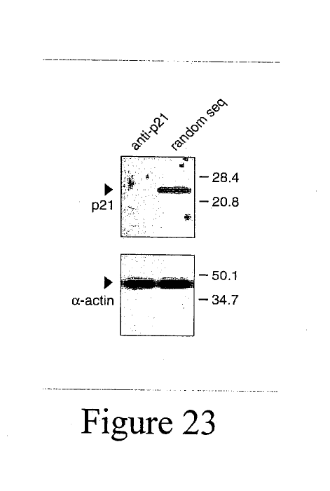

Using a random sequence phosphorothioate oligodeoxynucleotide (SEQ. ID NO.: 3) as a control, the effect of the antisense oligodeoxynucleotide to p21 (400 nM) on p21 and α-actin protein expression in A10 VSM cells, was examined. The lysates, normalized for protein content, were Western blotted with either p21 or α-actin antibody. There was marked inhibition of p21 protein level, but no effect on the level of α-actin protein level, after transfection of 400 nM antisense p21 oligodeoxynucleotide (SEQ. ID NO.: 1) (FIG. 23). Thus, this antisense oligodeoxynucleotide is specific in its inhibition of p21 protein expression and would not be expected to directly inhibit transcription or translation of matrix proteins which, of course, are unrelated in sequence to the CKIs.

1. Effect of Transfection of Antisense p21 (SEQ. ID NO.: 1) or Random Sequence (SEQ. ID NO.: 3) Oligodeoxynucleotide on TGF-β

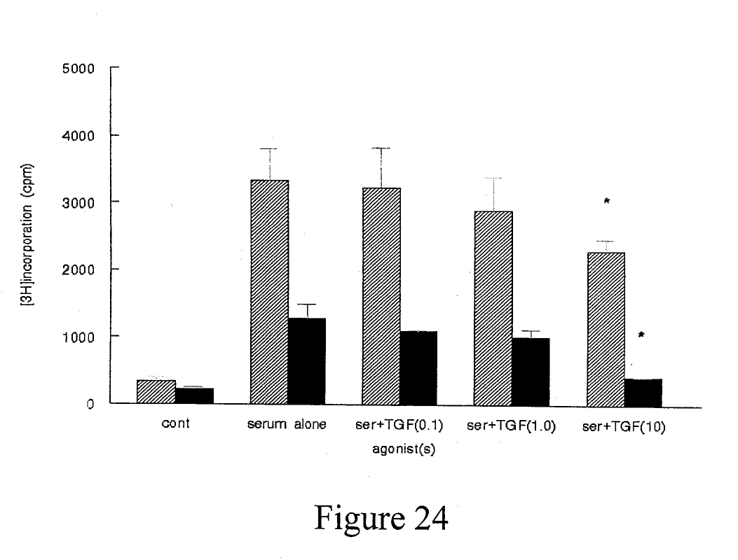

Cells were transfected with the appropriate oligodeoxynucleotide (antisense or random sequence) for 4 h. The cells were then serum-starved overnight and subsequently stimulated with 10% serum-containing media. Two hours after stimulation with serum, TGF-β was added at concentrations from 0.1 to 10 ng/ml for 24 h. While DNA synthesis of cells transfected with antisense p21 was markedly inhibited relative to cells transfected with control oligodeoxynucleotide (as it was previously shown when comparing antisense p21 with sense p21 oligodeoxynucleotide (Example 1, herein), the inhibitory effect of TGF-β at 10 ng/ml was still present under both conditions (FIG. 24).

2. Effect of p21 on Matrix Protein Secretion in VSM Cells

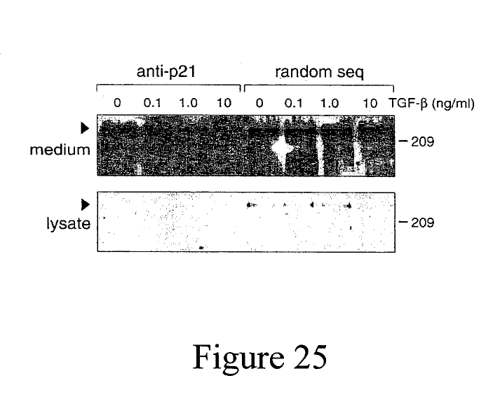

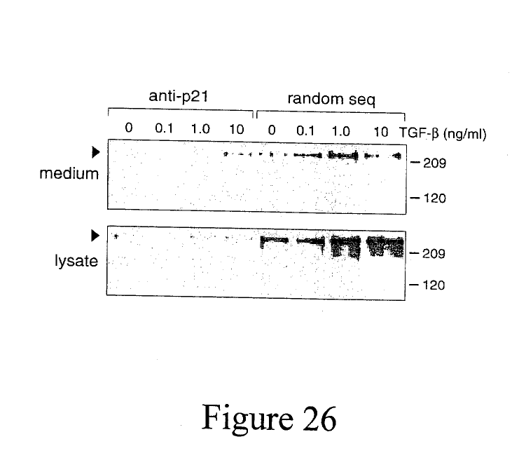

The p21 influences on matrix protein secretion in VSM cells were determined. Levels of the matrix proteins laminin and fibronectin were examined in both TGF-β stimulated lysate and conditioned media of cells in which p21 expression had been attenuated. The cells were transfected with either antisense p21 (SEQ. ID NO.: 1) (400 nM), or random sequence control (SEQ. ID NO.: 3) (400 nM) oligodeoxynucleotide, and then stimulated with TGF-β at for 0.1 to 10 ng/ml for 4 h. Conditioned media and cell lysate were collected, and both medium and lysate volumes were normalized for the protein content in total cell lysate to exclude any skewing of the data due to cell proliferation. The proteins were electrophoresed and immunoblotted with either fibronectin (after immunoprecipitation to eliminate extraneous bands which appeared in the absence of this procedure) or laminin antibody. Production and secretion of laminin was markedly reduced after attenuation of p21, yet the effect of TGF-β was to decrease laminin secretion at higher doses in control oligodeoxynucleotide transfected cells (FIG. 25). Fibronectin production and secretion into the medium was similarly decreased after p21 attenuation, yet, in this case, TGF-β induced fibronectin production with a maximal level in lysate when fibronectin was administered at higher doses (FIG. 26).

Discussion

The early lesions of atherosclerosis are associated with migration and proliferation of VSM cells. Once these cells enter the proliferative state, they attain a synthetic phenotype which causes them to secrete matrix proteins (Assoian, R. K.; Marcantonio, E. E. J. Clin. Invest 100: S15-S18; 1997; and Thyberg, J.; et al. Arteriosclerosis 10: 966-990; 1990). Exuberant secretion of these proteins may lead to fibrosis, but the same proteins may also regulate the cell phenotype and cause it to either remain secretory or become proliferative (Thyberg, J et al. J. Histochem. Cytochem. 45: 837-846; 1997).

Matrix proteins are secreted by a variety of cells and are important for structural integrity in the normal environment, yet these same proteins may be detrimental when they occur in abundance in the disease setting (reviewed in (Rizzino, A. Dev. Biol. 130: 411-422; 1988)). Furthermore, matrix proteins have been assigned the role of cell cycle control elements in atherosclerotic disease (Assoian, R. K.; Marcantonio, E. E. J. Clin. Invest 100: S15-S18; 1997). The specific matrix proteins laminin and fibronectin are important in modulating the switch from contractile to synthetic phenotype in VSM cells (reviewed in (Thyberg, J.; et al. Arteriosclerosis 10: 966-990; 1990)).

The extracellular matrix plays a key role in the progression of fibrosis in a variety of disparate diseases in multiple organ systems. In VSM and related glomerular mesangial cells, overexuberant secretion of matrix proteins is likely responsible for progression of atherosclerosis as well as of glomerular disease (reviewed in (Assoian, R. K.; Marcantonio, E. E. J. Clin. Invest 100: S15-S18; 1997; and Border, W. A.; Noble, N. A. N. Engl. J. Med. 331: 1286-1292; 1994)). The growth factor TGF-β, which in VSM cells is generally growth inhibitory (Weiss, R. H.; et al. Kidney Int. 48: 738-744; 1995; and Reddy, K. B.; Howe, P. H. J. Cell. Physiol. 156: 48-55; 1993), causes fibrosis in a variety of tissues and has been linked to an increase in matrix protein production as an etiology for this pathologic process (Border, W. A.; et al. Kidney Int. 37: 689-695; 1990; and Nakamura, T.; et al. Kidney Int. 41: 1213-1221; 1992), such that antibodies to TGF-β1 suppress arterial intimal hyperplasia and restenosis (Wolf, Y. G.; et al. J. Clin. Invest 93: 1172-1178; 1994) as well as experimental glomerulonephritis (Border, W. A.; et al. Nature 346: 371-374; 1990).

Studies have shown that the CKI p27 may mediate the switch from hyperplasia to the hypertrophic phenotype in VSM cells in response to TGF-β (Gibbons, G. H.; et al. J. Clin. Invest. 90: 456-461; 1992 Braun-Dullaeus, R. C.; et al. J. Clin. Invest. 104: 815-823; 1999)). Further, CKIs are important in regulating cell cycle transit (Sherr, C. J.; Roberts, J. M. Genes and Dev. 13: 1501-1512; 1999). Thus it was decided to examine whether the CKI p21 plays a role in the regulation of TGF-β-mediated matrix protein synthesis and secretion in VSM cells.

TGF-β is a growth factor that has variable influences on VSM and glomerular mesangial cells, the latter of which are modified smooth muscle cells. Depending on the cell type and culture conditions, TGF-β can be either stimulatory or inhibitory towards cell growth (Moses, H. L.; et al. Cell 63: 245-247; 1990). Despite its bimodal effect on cell proliferation, it is clear that TGF-β induces the pathologic appearance of matrix proteins, and thus this growth factor has been implicated as a causative agent in a variety of diseases which are characterized by fibrosis (Border, W. A.; Noble, N. A. N. Engl. J. Med. 331: 1286-1292; 1994; and Border, W. A.; et al. Kidney Int.Suppl 49: S59-S61; 1995). There is even evidence that fibronectin (Madri, J. A.; et al. J.Cell Biol. 106: 1375-1384; 1988) and laminin (Thyberg, J et al. J Histochem. Cytochem. 45: 837-846; 1997) may actually be mediating the growth inhibitory effects of TGF-β. In any case, an understanding of the mechanism by which vascular cells attain the secretory phenotype, and can therefore be influenced by TGF-β to secrete matrix proteins, is of pivotal importance in the study of fibrotic diseases.

The results described herein show that p21 antisense oligodeoxynucleotides, which specifically inhibit p21 protein levels in these cells, markedly attenuate both synthesis and secretion of the matrix proteins fibronectin and laminin. These results in vascular cells have profound implications for treatment of fibrotic diseases, such as atherosclerosis and restenosis. Furthermore, since glomerular mesangial cells are modified VSM cells, the findings can be extended to treatment of glomerular diseases characterized by fibrotic changes mediated through matrix protein production.

The CKIs p21 and p27 have long been known to be induced by TGF-β (Polyak, K.; et al. Genes Dev. 8: 9-22; 1994; and Grau, A. M.; et al. Cancer Res. 57: 3929-3934; 1997), and it has been assumed that expression of those proteins links this growth factor to cell cycle arrest. It has been shown that in VSM, mesangial, and prostate carcinoma cells, p27 and p21 are induced by the growth inhibitory statins (Weiss, R. H.; Ramirez, A.; Joo, A. J. Am. Soc. Nephrol. 9: 1880-1890; 1999; Lee, S. J.; et al. J. Biol. Chem. 273: 10618-10623; 1998; and Terada, Y.; et al. J. Am. Soc.Nephrol. 9: 2235-2243; 1999). While these proteins likely in some manner cause attenuation of DNA synthesis in that setting, it has also been shown that p21 is required for the full mitogenic effect of PDGF and serum (Weiss, R. H.; et al. J. Biol. Chem. 275: 10285-10290; 2000; Weiss, R. H. and Randour, C. Cellular Signalling, 12:413-418, 2000), a correlation which has recently been confirmed by another group (Wakino, S, et al. J Biol Chem 275(9):22435-22441,2000). Other investigators have shown the existence of a switch from a contractile to a synthetic phenotype in cells stimulated to proliferate (Thyberg, J.; et al. Arteriosclerosis 10: 966-990; 1990). The data presented in Example IV are consistent with this scenario, since decreased synthesis of matrix protein in cells transfected with antisense p21 was shown, concomitant with attenuation of proliferation in these cells.

Example IV demonstrates that the ability of TGF-β to synthesize and secrete the matrix proteins laminin and fibronectin, but not TGF-β's entire growth inhibitory effect, is mediated through p21. It was previously shown that, in mesangial cells, TGF-β at 10 ng/ml induced secretion of the matrix proteins fibronectin and laminin (Weiss, R. H.; Ramirez, A. Nephrol. Dial. Transplant. 13: 2804-2813; 1998). However, in the VSM cells used in the present experiments, higher concentrations of TGF-β were associated with decreased laminin synthesis and secretion into conditioned media. This may be due to the fact that continued stimulation of VSM cells by TGF-β causes them to remain in a synthetic phenotype associated with fibronectin secretion, whereas cells remain in the contractile phenotype when grown in the presence of the secreted laminin (Thyberg, J et al. J. Histochem. Cytochem. 45: 837-846; 1997).

It is noteworthy that transfection of antisense oligodeoxynucleotides has been shown to be effective in vivo both with and without lipofection reagents, and in some situations antisense oligodeoxynucleotides have even proved effective under conditions as simple as intravenous oligodeoxynucleotide infusions (Monia, B. P.; et al. Nat. Med. 2: 668-675; 1996). Therefore, these compounds may be useful clinically to target molecules important in fibrotic diseases such as, atherosclerosis, restenosis and glomerular disease.

It has been demonstrated that, in the case of mesangial cells, p21 is required for glomerular hypertrophy in experimental diabetic nephropathy (Al Douahji, M.; et al. Kidney Int. 56: 1691-1699; 1999). p21 maybe allowing secretion of matrix proteins in this scenario such that its inhibition would diminish this response. In VSM cells, p27 has been shown to have a similar role in the promotion of hypertrophy (Braun-Dullaeus, R. C.; et al. J. Clin. Invest. 104: 815-823; 1999), yet the role of p21 and matrix protein secretion in this phenomenon in VSM cells is not known. In the setting-of angioplasty, VSM cells have been shown to modulate from a contractile to a synthetic phenotype after induction of intimal lesions by balloon catheterization (Grunwald, J.; et al. Exp. Mol. Pathol. 46: 78-88; 1987; and Manderson, J. A.; et al. Arteriosclerosis 9: 289-298; 1989). This may in turn result in excess production of matrix proteins, leading to ultimate restenosis of the vessel. Attenuation of matrix protein production and secretion with antisense p21 transfection into VSM cells may therefore prove to be useful, where such oligodeoxynucleotides could be lipofected into angioplastied blood vessels at the time of balloon catheterization.

A study was performed in cells from knockout mice, where it was shown that lack of a functional p21 gene ameliorated progression to chronic renal failure (Megyesi, J.; et al. Proc. Natl. Acad. Sci.U.S.A. 96: 10830-10835; 1999). In this study, none of the p21(−/−) mice developed glomerulosclerosis or interstitial fibrosis, as opposed to 70% of the glomeruli in p21(+/+) animals, suggesting that p21 may be responsible for mediating a TGF-β effect on the cells leading to fibrosis by means of matrix protein secretion.

### EXAMPLE V

### In Vivo Studies of the Effect of Antisense Oligonucletide of p21 on Met-1 Breast Cancer

Angiogenesis is the means by which growing tumors maintain oxygen necessary for their survival by means of their creation of auxiliary blood vessels. This is generally thought to result for the effect of various growth factors (such as VEGF) acting on endothelial cells. The possibility that angiogenesis may also be attenuated by inhibition of VSM cells is a possibility which has not been adequately investigated.

Materials and Methods: NZW mice were obtained and injected subcutaneously 2 days after their arrival with Met-1 breast cancer cells. Each mouse was injected on two sides of the breast area, such that two tumors arose in most animals. The following day, intraperitineal injections at the indicated concentrations were made on a daily basis. When tumors appeared, they were measured in two dimensions using calipers and area of the tumors was used as rough measure of tumor mass. The tumor areas were averaged for each mouse and then these numbers were subsequently averaged. The general health of the mice was monitored on a daily bases as well.

Antisense Preparation: Phosphorothioate antisense oligodeoxynucleotides were synthesized by Oligonucleotides Etc. (Wilsonville, Oreg.). The p21^{Waf1/Cip1} antisense vector was designed around the start codon of rat p21^{Waf1/Cip1}, with sequence 5′-GAC ATC ACC AGG ATC GGA CAT-3′ (SEQ. ID NO.: 1). The sense p21^{Waf1/Cip1} sequence is 5′-ATG TCC GAT CCT GGT GAT GTC-3′ (SEQ. ID NO.: 2). The scrambled random sequence control oligodeoxynucleotide was 5′-TGG ATC CGA CAT GTC AGA-3′ (SEQ. ID NO.: 3).

Animal models of potential chemotherapeutics using the p21 antisense oligonucleotides of the invention were examined. Intraperitoneal injection of the oligonucleotides into mice previously injected with cells from a Met-1 breast cancer line (Bourguignon, L. Y. et al, J. Cell Phys. 1998, 176, 206-215; Lau, D. H. et al., Cancer Biother. Radiopharm. 1999, 14, 31-6) were studied. The p21 antisense oligonucleotide or random sequence control oligonucleotide at a concentration of 0.6 mg oligonucleotide/kg mouse (Monia, B. P.; et al. , 1996, Nat. Med. 2: 668-675)were injected intraperitoneally daily when tumors first became palpable (day 1). There were 2 tumors per mouse (palpable in the breast area), and the tumors were measured in 2 dimensions by calipers at the times indicated. The areas of the 2 tumors per mouse were averaged and then these numbers were averaged over all 3 mice per data point and presented as the mean+/−s.e.m. The results are shown in FIG. 27. Transfection of the antisense p21 oligonucleotides did not affect growth of Met-1 cells in culture, suggesting an anti-angiogenesis effect in vivo.

What is claimed is:

1. A method for inhibiting cyclin-dependent kinase-mediated cell growth and proliferation, comprising inhibiting p21^{Waf1/Cip1} protein with a p21^{Waf1/Cip1} inhibitory agent in an amount effective to inhibit cell growth and proliferation.

2. The method of claim 1, wherein said p21^{Waf1/Cip1} inhibitory agent is an antisense oligonucleotide molecule directed to the nucleotide sequence of p21^{Waf1/Cip1}.

3. The method of claim 1, wherein said p21^{Waf1/Cip1} inhibitory agent is an antibody directed against the p21^{Waf1/Cip1} protein.

4. The method of claim 3, wherein the antibody is a monoclonal antibody.

5. The method of claim 3, wherein the monoclonal antibody comprises murine antigen binding region residues and human antibody residues.

6. The method of claim 3, wherein the monoclonal antibody is a humanized antibody.

7. The method of claim 3, wherein the monoclonal antibody is a human antibody.

8. A method of inhibiting a disease associated with abnormal cell growth and proliferation, comprising inhibiting p21^{Waf1/Cip1} protein by administering a p21^{Waf1/Cip1} inhibitory agent in an amount effective to inhibit cell growth and proliferation.

9. The method of claim 8, wherein said p21^{Waf1/Cip1} inhibitory agent is an antisense oligonucleotide molecule directed to the nucleotide sequence of p21^{Waf1/Cip1}.

10. The method of claim 8, wherein said p21^{Waf1/Cip1} inhibitory agent is an antibody directed against the p21^{Waf1/Cip1} protein.

11. The method of claim 10, wherein the antibody is a monoclonal antibody.

12. The method of claim 10, wherein the monoclonal antibody comprises murine antigen binding region residues and human antibody residues.

13. The method of claim 10, wherein the monoclonal antibody is a humanized antibody.

14. The method of claim 10, wherein the monoclonal antibody is a human antibody.

15. The method of claim 8, wherein the disease is a fibrotic disease.

16. The method of claim 15, wherein the disease is selected from the group comprising of atherosclerosis, angioplasty restenosis, and renal mesangial cell proliferation.

17. The method of claim 8, wherein the disease is cancer.

18. The method of claim 8, further comprising exposing the cells to radiation.

19. The method of claim 8, further comprising administering a chemotherapeutic drug.

20. A method of inhibiting angiogenesis, comprising inhibiting p21^{Waf1/Cip1} protein by administering a p21^{Waf1/Cip1} inhibitory agent in an amount effective to inhibit angiogensis in tumors.

21. The method of claim 20, wherein said p21^{Waf1/Cip1} inhibitory agent is an antisense oligonucleotide molecule directed to the nucleotide sequence of p21^{Waf1/Cip1}.

22. The method of claim 20, wherein said p21^{Waf1/Cip1} inhibitory agent is an antibody directed against the p21^{Waf1/Cip1} protein.

23. The method of claim 22, wherein the antibody is a monoclonal antibody.

24. The method of claim 22, wherein the monoclonal antibody comprises murine antigen binding region residues and human antibody residues.

25. The method of claim 22, wherein the monoclonal antibody is a humanized antibody.

26. The method of claim 22, wherein the monoclonal antibody is a human antibody.

27. A method of inhibiting the growth of tumor cells, comprising administering to a patient an p21^{Waf1/Cip1} inhibitory agent in an amount effective to inhibit growth of the tumor cells.

28. The method of claim 27, wherein said p21^{Waf1/Cip1} inhibitory agent is an antisense oligonucleotide molecule directed to the nucleotide sequence of p21^{Waf1/Cip1}.

29. The method of claim 27, wherein said p21^{Waf1/Cip1} inhibitory agent is an antibody directed against the p21^{Waf1/Cip1} protein.

30. The method of claim 29, wherein the antibody is a monoclonal antibody.

31. The method of claim 29, wherein the monoclonal antibody comprises murine antigen binding region residues and human antibody residues.

32. The method of claim 29, wherein the monoclonal antibody is a humanized antibody.

33. The method of claim 29, wherein the monoclonal antibody is a human antibody.

34. The method of claim 27, further comprising administering a chemotherapeutic drug.

35. The method of claim 27, further comprising administering radiation therapy.

36. A pharmaceutical composition comprising a p21^{Waf1/Cip1} inhibitory agent.

37. The composition of claim 36, wherein said inhibitory agent is an antisense oligonucleotide molecule directed to the nucleotide sequence of p21^{Waf1/Cip1}.

38. The composition of claim 36, wherein said inhibitory agent is an antibody directed against the p21^{Waf1/Cip1}.

39. The composition of claim 38, wherein said antibody is a monoclonal antibody.


SEQUENCE LISTING (3 sequences)

SEQ ID NO: 1 - DNA, 21 bp, Artificial Sequence
  Description of Artificial Sequence antisense p21Waf1/Cip1 oligodeoxynucleotide
gacatcaccaggatcggacat

SEQ ID NO: 2 - DNA, 21 bp, Artificial Sequence
  Description of Artificial Sequence sense p21Waf1/Cip1 oligodeoxynucleotide
atgtccgatcctggtgatgtc

SEQ ID NO: 3 - DNA, 18 bp, Artificial Sequence
  Description of Artificial Sequence random sequence oligodeoxynucleotide
tggatccgacatgtcaga